SEQUENCE LISTING (76 sequences)

SEQ ID NO: 1 - DNA, 20 bp, artificial sequence
  beta-actin forward primer
atcatgtttgagaccttcaa

SEQ ID NO: 2 - DNA, 22 bp, artificial sequence
  beta-actin reverse primer
tctgcgcaagttaggttttgtc

SEQ ID NO: 3 - DNA, 21 bp, artificial sequence
  beta3-tubulin forward primer
ctcagtcctagatgtcgtgcg

SEQ ID NO: 4 - DNA, 20 bp, artificial sequence
  beta3-tubulin reverse primer
gcggaagcagatgtcgtaga

SEQ ID NO: 5 - DNA, 23 bp, artificial sequence
  beta2/neuroD forward primer
ctgatctggtctccttcgtacag

SEQ ID NO: 6 - DNA, 21 bp, artificial sequence
  beta2/neuroD reverse primer
gatgcgaatggctatcgaaag

SEQ ID NO: 7 - DNA, 20 bp, artificial sequence
  brachyury forward primer
agtatgaacctcggattcac

SEQ ID NO: 8 - DNA, 20 bp, artificial sequence
  brachyury reverse primer
ccggttgttacaagtctcag

SEQ ID NO: 9 - DNA, 21 bp, artificial sequence
  GATA-1 forward primer
gggaagagcaacaacacgttc

SEQ ID NO: 10 - DNA, 21 bp, artificial sequence
  GATA-1 reverse primer
gtttgctgacaatcattcgct

SEQ ID NO: 11 - DNA, 20 bp, artificial sequence
  GATA-4 forward primer
agcctacatggccgacgtgg

SEQ ID NO: 12 - DNA, 20 bp, artificial sequence
  GATA-4 reverse primer
tcagccaggaccaggctgtt

SEQ ID NO: 13 - DNA, 22 bp, artificial sequence
  GFAP forward primer
agaacaacctggctgcgtatag

SEQ ID NO: 14 - DNA, 23 bp, artificial sequence
  GFAP reverse primer
tctgcaaacttagaccgatacca

SEQ ID NO: 15 - DNA, 20 bp, artificial sequence
  glucokinase forward primer
cacccaactgcgaaatcacc

SEQ ID NO: 16 - DNA, 20 bp, artificial sequence
  glucokinase reverse primer
catttgtggggtgtggagtc

SEQ ID NO: 17 - DNA, 19 bp, artificial sequence
  Glut2 forward primer
ccacccagtttacaagctc

SEQ ID NO: 18 - DNA, 20 bp, artificial sequence
  Glut2 reverse primer
tgtaggcagtacgggtcctc

SEQ ID NO: 19 - DNA, 22 bp, artificial sequence
  Hlbx9 forward primer
acaagtacctgtctcgacccaa

SEQ ID NO: 20 - DNA, 21 bp, artificial sequence
  Hlbx9 reverse primer
ctcagatgagcagtcggatga

SEQ ID NO: 21 - DNA, 18 bp, artificial sequence
  HNF3beta forward primer
cagaccacgcgagtccta

SEQ ID NO: 22 - DNA, 22 bp, artificial sequence
  HNF3beta reverse primer
catgatccactgatagatctcg

SEQ ID NO: 23 - DNA, 19 bp, artificial sequence
  Ins2 forward primer
ccctgctggccctgctctt

SEQ ID NO: 24 - DNA, 20 bp, artificial sequence
  Ins2 reverse primer
aggtctgaaggtcacctgct

SEQ ID NO: 25 - DNA, 24 bp, artificial sequence
  IsI-I forward primer
tctaaggtgtaccacatcgagtgt

SEQ ID NO: 26 - DNA, 21 bp, artificial sequence
  IsI-I reverse primer
gcaggctgatctatgtcgctt

SEQ ID NO: 27 - DNA, 21 bp, artificial sequence
  MAP2 forward primer
aaggccaagaacacacgattg

SEQ ID NO: 28 - DNA, 23 bp, artificial sequence
  MAP2 reverse primer
accaagccctaagcttcgactaa

SEQ ID NO: 29 - DNA, 24 bp, artificial sequence
  MashI forward primer
aagaagatgagcaaggtggagacg

SEQ ID NO: 30 - DNA, 24 bp, artificial sequence
  MashI reverse primer
cagaaccagttggtaaagtccagc

SEQ ID NO: 31 - DNA, 21 bp, artificial sequence
  nanog forward primer
gcaccaactcaacttctgagc

SEQ ID NO: 32 - DNA, 21 bp, artificial sequence
  nanog reverse primer
ctcgagagtagccaccatatc

SEQ ID NO: 33 - DNA, 20 bp, artificial sequence
  nestin forward primer
atacaggactctgctggagg

SEQ ID NO: 34 - DNA, 20 bp, artificial sequence
  nestin reverse primer
aggacaccagtagaactggg

SEQ ID NO: 35 - DNA, 21 bp, artificial sequence
  NgnI forward primer
tgcatctctgatctcgactgc

SEQ ID NO: 36 - DNA, 22 bp, artificial sequence
  NgnI reverse primer
agatgtagttgtaggcgaagcg

SEQ ID NO: 37 - DNA, 21 bp, artificial sequence
  Ngn2 forward primer
aagctcacgaagatcgagacg

SEQ ID NO: 38 - DNA, 21 bp, artificial sequence
  Ngn2 reverse primer
tgccagtagtccacgtctgac

SEQ ID NO: 39 - DNA, 21 bp, artificial sequence
  Ngn3 forward primer
catacctagggactgctccga

SEQ ID NO: 40 - DNA, 21 bp, artificial sequence
  Ngn3 reverse primer
catacaagctgtggtccgcta

SEQ ID NO: 41 - DNA, 20 bp, artificial sequence
  Nkx2.2 forward primer
ctcttctccaaagcgcagac

SEQ ID NO: 42 - DNA, 20 bp, artificial sequence
  Nkx2.2 reverse primer
aacaaccgtggtaaggatcg

SEQ ID NO: 43 - DNA, 22 bp, artificial sequence
  Nkx6.1 forward primer
ttctctggacagcaaatcttcg

SEQ ID NO: 44 - DNA, 22 bp, artificial sequence
  Nkx6.1 reverse primer
ctgagtgattttctcgtcgtca

SEQ ID NO: 45 - DNA, 20 bp, artificial sequence
  Oct4 forward primer
gaaagcaactcagagggaac

SEQ ID NO: 46 - DNA, 20 bp, artificial sequence
  Oct4 reverse primer
cttctctagcccaagctgat

SEQ ID NO: 47 - DNA, 20 bp, artificial sequence
  Olig2 forward primer
gtgtctagtcgcccatcgtc

SEQ ID NO: 48 - DNA, 21 bp, artificial sequence
  Olig2 reverse primer
tctttcttggtggaagacgtg

SEQ ID NO: 49 - DNA, 22 bp, artificial sequence
  p48/PtfI forward primer
agccaccagctacacgaatact

SEQ ID NO: 50 - DNA, 21 bp, artificial sequence
  p48/PtfI reverse primer
gaggagggagaccataatccg

SEQ ID NO: 51 - DNA, 21 bp, artificial sequence
  p75 forward primer
gtgcggggtgggctcaggact

SEQ ID NO: 52 - DNA, 22 bp, artificial sequence
  p75 reverse primer
ccacaaggcccacaaccacagg

SEQ ID NO: 53 - DNA, 24 bp, artificial sequence
  Pax3 forward primer
ggaggcggatctagaaaggaagga

SEQ ID NO: 54 - DNA, 22 bp, artificial sequence
  Pax3 reverse primer
cccccggaatgagatggttgaa

SEQ ID NO: 55 - DNA, 21 bp, artificial sequence
  Pax4 forward primer
gtgagcaagatcctaggacgc

SEQ ID NO: 56 - DNA, 21 bp, artificial sequence
  Pax4 reverse primer
cggggagaagatagtccgatt

SEQ ID NO: 57 - DNA, 20 bp, artificial sequence
  Pax6 forward primer
tcacagcggagtgaatcagc

SEQ ID NO: 58 - DNA, 22 bp, artificial sequence
  Pax6 reverse primer
tatcgttggtacagaccccctc

SEQ ID NO: 59 - DNA, 20 bp, artificial sequence
  PDX-I forward primer
gacatctccccatacgaagt

SEQ ID NO: 60 - DNA, 22 bp, artificial sequence
  PDX-I reverse primer
gtcccgctactacgtttcttat

SEQ ID NO: 61 - DNA, 21 bp, artificial sequence
  slug forward primer
tactgtatggacatcgtcggc

SEQ ID NO: 62 - DNA, 23 bp, artificial sequence
  slug reverse primer
acagtacttgcagctgaacgatt

SEQ ID NO: 63 - DNA, 21 bp, artificial sequence
  snail forward primer
aagcccaactatagcgagctg

SEQ ID NO: 64 - DNA, 21 bp, artificial sequence
  snail reverse primer
agttgaagatcttccgcgact

SEQ ID NO: 65 - DNA, 19 bp, artificial sequence
  SoxI forward primer
aatcccctctcagacggtg

SEQ ID NO: 66 - DNA, 19 bp, artificial sequence
  SoxI reverse primer
ttgatgcattttgggggta

SEQ ID NO: 67 - DNA, 21 bp, artificial sequence
  Sox2 forward primer
ggagtggaaacttttgtccga

SEQ ID NO: 68 - DNA, 21 bp, artificial sequence
  Sox2 reverse primer
ttcatgtaggtctgcgagctg

SEQ ID NO: 69 - DNA, 21 bp, artificial sequence
  Sox3 forward primer
agactgaactcaagaaccccg

SEQ ID NO: 70 - DNA, 21 bp, artificial sequence
  Sox3 reverse primer
gtccttcttgagcagcgtctt

SEQ ID NO: 71 - DNA, 21 bp, artificial sequence
  Sox10 forward primer
ggaggttgctgaacgaaagtg

SEQ ID NO: 72 - DNA, 21 bp, artificial sequence
  Sox10 reverse primer
tccatgttggacattacctcg

SEQ ID NO: 73 - DNA, 24 bp, artificial sequence
  twist forward primer
ctttccgcccacccacttcctctt

SEQ ID NO: 74 - DNA, 24 bp, artificial sequence
  twist reverse primer
gtccacgggcctgtctcgctttct

SEQ ID NO: 75 - DNA, 21 bp, artificial sequence
  WntI forward primer
tgacaacatcgattttggtcg

SEQ ID NO: 76 - DNA, 21 bp, artificial sequence
  WntI reverse primer
ggcgatttctcgaagtagacg

(19) United States
(12) Patent Application Publication
(10) Pub. No.: US 2005/0277190 A1
(43) Pub. Date: Dec. 15, 2005

(54) Pancreatic stem cells

(76) Inventors: Raewyn Seaberg, Toronto (CA); Simon Smukler, Thornhill (CA); Derek Van Der Kooy, Toronto (CA)

Correspondence Address: BERESKIN AND PARR, 40 KING STREET WEST, BOX 401, TORONTO, ON, M5H 3Y2, CA

(21) Appl. No.: 11/072,630

(22) Filed: Mar. 7, 2005

Related U.S. Application Data
(60) Provisional application No. 60/550,056, filed on Mar. 5, 2004.

(30) Foreign Application Priority Data
Mar. 10, 2004 (CA) 2,460,602

(51) Int. Cl. C12N 5/08

(52) U.S. Cl. 435/366; 435/368

(57) ABSTRACT

Pancreatic progenitor cells isolated from the pancreas of a mammal. The invention also includes pancreatic cells or neural cells differentiated from the pancreatic progenitor cells.

### CROSS REFERENCE TO RELATED APPLICATION

This application claims priority from U.S. provisional application No. 60/550,056, filed on Mar. 5, 2004, which is incorporated by reference herein in its entirety.

### FIELD OF THE INVENTION

The invention relates to stem cells isolated from the pancreatic islet- and duct-derived tissue of mammals. The invention includes a method for stimulating proliferation of endogenous pancreatic stem cells in vivo and pharmaceutical compounds that stimulate proliferation of pancreatic stem cells. The invention also relates to a method for isolating pancreatic stem cells, a method for passaging isolated clonal pancreatic stem cells, uses for the clonally isolated stem cells and pharmaceutical compositions containing the stem cells or their progeny. The invention can be used to treat individuals having pancreatic diseases (such as diabetes), disorders or abnormal physical states. The invention includes pancreatic stem cells and pancreatic cell culture systems for toxicological assays, drug development, isolating genes involved in pancreatic differentiation or for developing tumor cell lines. The invention also includes pancreatic multipotent progenitor (PMP) cells isolated from the pancreatic islet- and duct-derived tissue of mammals and methods of using the progenitor cells.

### BACKGROUND OF THE INVENTION

There have been no reports to date of the clonal isolation and proliferation of single adult pancreatic precursor cells. These cells would be useful as a potential source of β-cells for transplantation in the treatment of diabetes. There is currently no way to reverse permanent damage to the pancreas. Drug treatments focus on treating the illness and its symptoms to prevent further damage to the pancreas. There is a need to reverse damage to the pancreas and restore function by endogenously generating new pancreatic cells or transplanting pancreatic cells. As such, the primary focus of many studies has been to develop strategies for the derivation and expansion of insulin-producing β-cells and other islet cell types². A number of issues relating to the nature of pancreatic precursors have not been fully resolved, including whether the cells possess the properties of a stem or progenitor cell²⁵, whether they reside in the islets of the endocrine pancreas or in the ducts of the exocrine pancreas, and what their full lineage potential might be^{3,4,5}.

One of the main obstacles to investigating these types of issues is that there have been no reports to date of the clonal isolation and proliferation of single adult pancreatic precursor cells. Several patents/patent applications have described the isolation of pancreatic progenitor cells in general (U.S. 20040058398; U.S. 20020192816; U.S. Pat. Nos. 6,703,017; 6,610,535; U.S. 20040005301), however, none of the prior art references have clonally isolated pancreatic progenitor cells. There are several advantages to having a clonal population of progenitor cells. Firstly, the negative effects of contaminating populations can be avoided. For example, bulk populations of mixed cells make it much more difficult to determine the direct effects on stem cells from indirect effects on other cells. Secondly, it is much easier to apply drugs or manipulate genes using clonal stem cells. It is desirous to have clonal stem cells since it is only stem cells that will have long-term self-renewal in vivo after transplantation or endogenous activation. Clonal stem cells could also be used to create a cell line that could be characterized and used indefinitely, for example, in assays or transplantation.

There are only two stem cell populations that currently are used clinically—blood and skin stem cells. Both cases demonstrate the need for clonally isolated stem cells. For example, the early bone marrow transplants used whole bone marrow. However, for patients with leukemia progenitor cell cancers, it would be very useful to autologously transplant more purified stem cells. With skin stem cells, the cells are expanded in culture for a long time in order to generate enough cells to do skin transplants in burn patients.

Therefore, there remains a need to identify single proliferative cells from the adult pancreas and characterize them in terms of their gene expression, their lineage potential, and their possible developmental origins.

Stem cells are undifferentiated cells that exist in many tissues of embryos and adult mammals. In embryos, blastocyst stem cells are the source of cells which differentiate to form the specialised tissues and organs of the developing fetus. In adults, specialised stem cells in individual tissues are the source of new cells which replace cells lost through cell death due to natural attrition, disease or injury. Additional information about stem cells is found, for example in U.S. Pat. No. 6,117,675.

Stem cells are capable of producing either new stem cells or cells called progenitor cells that differentiate to produce the specialised cells found in mammalian organs. Symmetric division occurs where one stem cell divides into two daughter stem cells. Asymmetric division occurs where one stem cell forms one new stem cell and one progenitor cell.

A progenitor cell differentiates to produce the mature specialized cells of mammalian organs. In contrast, stem cells never terminally differentiate (i.e. they never differentiate into a specialized tissue cells). Progenitor cells and stem cells are referred to collectively as “precursor cells”. This term is used when it is unclear whether a researcher is dealing with stem cells or progenitor cells or both.

Progenitor cells may differentiate in a manner which is unipotential or multipotential. A unipotential progenitor cell is one which can form only one particular type of cell when it is terminally differentiated. A multipotential progenitor cell has the potential to differentiate to form more than one type of tissue cell. Which type of cell it ultimately becomes depends on conditions in the local environment as such as the presence or absence of particular peptide growth factors, cell-cell communication, amino acids and steroids. For example, it has been determined that the hematopoietic stem cells of the bone marrow produce all of the mature lymphocytes and erythrocytes present in fetuses and adult mammals. There are several well-studied progenitor cells produced by these stem cells, including three unipotential and one multipotential tissue cell. The multipotential progenitor cell may divide to form one of several types of differentiated cells depending on which hormones act upon it.

There is great potential for the use of progenitor cells and stem cells as substrates for producing healthy tissue where pathological conditions have destroyed or damaged normal tissue. For example, stem cells may be used as a target for in vivo stimulation with growth factors or they may be used as a source of cells for transplantation.

It would be useful if stem cells could be identified and isolated in areas of the pancreas. Medical treatments could then be developed using those stem cells.

For example, there remains a need for clonally isolated pancreatic cells for transplantation in which (1) the composition is accepted by the patient, thus avoiding the difficulties associated with immunosuppression, (2) the composition is safe and effective, thus justifying the cost and effort associated with treatment, (3) the composition provides long term relief of the symptoms associated with the disease, and (4) the composition is efficacious during and after transplantation. There is a clear need to develop pancreatic progenitor cell cultures which can act as a source of cells that are transplantable in vivo in order to replace damaged tissue.

There is also a need for pancreatic stem cell cultures or pancreatic cell cultures which may be used in toxicity testing, drug development and to isolate new genes and metabolites involved in cell differentiation. There is also a need for pancreatic cell cultures which may be used to develop derivative cell lines, for studying cancer or other diseases, disorders or abnormal states.

### SUMMARY OF THE INVENTION

The present inventors have clonally isolated novel stem cells from the adult murine pancreas. The unique application of a serum-free colony-forming assay to pancreatic cells enabled the identification of a subpopulation of progenitor cells from each of pancreatic islet and ductal populations. Accordingly, the invention relates to clonally isolated stem cells from the pancreas that generate neural and pancreatic lineages. These cells can proliferate in vitro to form clonal colonies that co-express neural and pancreatic precursor markers. Upon differentiation, individual clonal colonies produce distinct populations of neurons and glial cells; pancreatic endocrine β-, α-, and δ-cells cells; pancreatic exocrine cells and pancreatic stellate cells. Moreover, the de novo generated β-cells demonstrate glucose-dependent Ca²⁺-responsiveness and insulin release. Pancreas colonies do not express markers of ES cells, nor genes suggestive of mesodermal or neural crest origins. These cells represent a novel adult intrinsic pancreatic stem cell population and represent a promising new candidate for cell-based therapeutic strategies.

Accordingly, the invention provides for pancreatic stem cells which are clonally isolated and purified from the pancreas. The stem cells are optionally from any animal, such as a mammal (eg. human or mouse). The source may be adult, child or embryonic. Pancreatic cells are then differentiated from the pancreatic stem cells. Pancreatic cells which are optionally produced from the stem cells include alpha cells, delta cells, beta cells and the other cells described below. Neural cells are optionally produced, such as neurons, glial cells and oligodendrocytes. The pancreatic stem cells may be transformed or transfected with a heterologous gene. The growth or differentiation of the pancreatic stem cells may be stimulated by a growth factor. Proliferation is also induced by administering genetically engineered cells which secrete growth factors into the pancreas. In one embodiment, the growth factors are epidermal growth factor and/or fibroblast growth factor. In another embodiment, self-renewal and proliferation are enhanced by the presence of BIO ((2′Z,3′E)-6-Bromoindirumbin-3′oxime).

The pancreatic stem cells may also be used as sources of transplantable tissue, as they can be removed from the donor and transplanted into a recipient either before or after differentiation into pancreatic cells. This invention also satisfies the needs outlined above in that the pancreatic stem cells of this invention (1) are accepted by the patient because they can be taken from the patient's own body, (2) are safe in that the patient is not receiving cells or tissue from another source, (3) are effective in that the pancreatic stem cells can be differentiated into pancreatic cells for implantation and survive during and after implantation, and (4) offer the potential to provide long term relief of the symptoms of conditions associated with loss of one or more pancreatic cell types.

The invention also provides cell cultures which may be used in toxicity testing, drug development, developing derivative cell lines and isolating new genes or proteins and metabolites involved in cell differentiation.

It is another object of the invention to provide a pharmaceutical composition for use in implant therapy consisting of the pancreatic stem cells and pancreatic cells in a pharmaceutically acceptable carrier, auxiliary or excipient. The invention includes the use of the cells of the invention for preparation of a medicament. The invention includes the use of the cells of the invention as a pharmaceutical substance and for treatment of diseases and disorders of the nervous system and pancreas as described herein. The invention also relates to a method of treating a disease, disorder or abnormal state of the pancreas or nervous system by stimulating proliferation of pancreatic stem cells. According to one embodiment of this invention, a growth factor is introduced to pancreatic stem cells. In the method, the disease may be one of neural damage or trauma, neural paralysis (e.g. spinal cord injury), Alzheimer's disease, Parkinson's disease, creutzfelt-jacob disease, pancreatic degeneration, diabetes, pancreatitis, and cancers of the pancreas. The cells are useful for treating any neural or pancreas disease or disorder that would benefit from cell transplant. An individual suffering from a degenerative disease, disorder or abnormal physical state of the pancreas or nervous system may also be treated by implanting the pancreatic stem cells or pancreatic cells into the pancreas of the individual.

Another object of the invention is to provide a method for isolating and purifying pancreatic stem cells from the pancreas of a mammal by taking a sample of the pancreas from the mammal, dissociating the sample into single cells, placing the cells in culture, isolating the cells which proliferate in culture and differentiating these cells into pancreatic cells or other cells.

In another embodiment of the invention, where the mammal is a human and is suffering from a disease, disorder or abnormal physical state of the pancreas, the method includes implanting the pancreatic stem cells or pancreatic cells differentiated from the pancreatic stem cells, into the pancreas of the human. Where the mammal is a human and is not suffering, from a disease, disorder or abnormal physical state of the pancreas, the method includes implanting the pancreatic stem cells or pancreatic cells differentiated from the pancreatic stem cells into a second human who is suffering from the disease, disorder or abnormal physical state. These approaches are adapted for use with the nervous system.

Another object of the invention is to provide a kit, containing at least one type of cell selected from a group consisting of the pancreatic stem cells and the pancreatic cells or neural cells and directions for use in treatment of disease or disorders. The kit may be used for the treatment of a disease, disorder or abnormal physical state of the pancreas or nervous system.

The cells of the invention may also be used in a method for identifying a substance which is toxic to pancreatic stem cells and pancreatic cells or neural cells, by introducing the substance to a pancreatic stem cell culture or a pancreatic or neural cell culture differentiated from a pancreatic stem cell culture, and determining whether the cell culture is adversely affected by the presence of the substance, is employed.

The cells of the invention may also be used in a method for identifying a pharmaceutical which may be used to treat a disease, disorder or abnormal state of the pancreas or nervous system, by introducing the pharmaceutical to a pancreatic stem cell culture or a pancreatic or neural cell culture differentiated from a pancreatic stem cell culture, and determining whether the culture is affected by the presence of the pharmaceutical.

The invention also includes a method of stimulating proliferation of pancreatic stem cells, by the addition of growth factors (EGF and FGF2). Accordingly, another aspect of the invention is a method of treating a disease, disorder or abnormal state of the pancreatic tissue or nervous system, by stimulating proliferation of pancreatic stem cells. The method of treatment may be used in treating a disease, disorder or abnormal physical state of the pancreas such as one selected from a group consisting of type I or type II diabetes, pancreatitis, pancreatic degeneration and cancers of the pancreas.

The invention also includes an isolated colony or sphere (a colony may be a sphere or another form of colony if cultured on a surface) comprising pancreatic stem cells and/or cells derived therefrom. The invention also includes an isolated pancreatic stem cell expressing one or more cell markers and/or one or more neural-specific mRNA molecules, as described herein, and being multipotent and having multilineage potential. The invention thus includes an isolated pancreatic stem cell or cell derived therefrom, and methods of producing a pre-selected cell type the aforementioned cells. These methods involve providing the cells in conditions as described herein, for example in a cell culture or transplanted into an animal, such as mammal, preferably a human.

The invention also includes a method for screening for modulators of pancreatic stem cell differentiation comprising:
a. culturing pancreatic stem cells under conditions that produce differentiation in the absence of the modulator;
b. detecting any differentiation of the cells and cell types generated, if any, in the presence of the modulator compared to differentiation and cell types generated in the absence of the modulator;
c. determining whether the modulator affects the differentiation of the cells.

The modulators optionally comprise any culturing conditions that may modulate cellular differentiation. The invention also includes a method for screening for differentiation factors of cellular development comprising:
a. culturing pancreatic stem cells in the presence of the differentiation factor;
b. allowing cells to differentiate;
c. detecting differentiation of the cells, if any.

The method optionally further comprises determining whether the differentiation of the cells comprises pancreatic cell or neural cell development. The invention also includes a method for screening for differentiation factors of cellular development comprising:
a. culturing the pancreatic stem cells in the presence of the differentiation factor.
b. detecting any differentiation of the cells.

The method optionally further comprises determining whether the cells differentiate into a homogenous uniform cell base, for example a neural cell base or pancreatic cell base. The cells of the invention are optionally cultured in a transplantation media.

The invention also comprises transplantation of pancreatic stem cells or cells derived therefrom into a subject and differentiating the cells into mature pancreatic and neural cells. The inventors transplant stem cells or cells derived therefrom into recipient mice and other mammals. The cells integrate into the mammals, for example, the beta cells produce insulin without immune rejection or abnormal cell development. The materials and methods employed for transplantation will be readily apparent to those skilled in the art of cellular transplantation at the time the application and are further described herein.

The invention also includes pancreatic multipotent progenitor (PMP) cells isolated from the pancreatic islet- and duct-derived tissue of mammals, cells derived from these PMP cells (eg. other PMP cells or neural or pancreatic cells differentiated from PMP cells), and methods of using the progenitor cells in methods of the invention described in this application.

### BRIEF DESCRIPTION OF THE DRAWINGS

Embodiments of the invention will now be described in relation to the figures in which:

FIG. 1. Pancreatic stem cell (PSC) colonies are formed from progenitors present in adult pancreatic islet and duct cell isolates, and express markers characteristic of both neural and pancreatic precursors. A. The frequency of PSC colonies from pancreatic islet and duct cell isolates is similar. The data are expressed as the mean number of colonies (+SEM; n=14 independent experiments) formed per 10,000 cells plated. Islet and ductal cell isolates do not contain significantly different numbers of PMPs (p>0.05). B. Light micrograph of a PSC colony; PSC colonies morphologically resemble adult brain-derived neurospheres although on average they have a smaller diameter. Scale bar, 50 μm. C. Light micrograph of a neurosphere. Scale bar, 50 μm. D. RT-PCR for neural and pancreatic precursor markers. The numbers on the left represent the number of individual PSC colonies that expressed the corresponding mRNA out of the total number of colonies tested by RT-PCR analysis. Note that nearly all PSC colonies assayed co-expressed the early pancreatic precursor marker PDX-1 and the neural precursor marker nestin. Further, multiple other neural and pancreatic precursor genes were expressed in clonal PSC colonies. Only single colony RNA isolates that were found to express β-actin were considered. Note that positive control (+) bands (see Supplementary Methods for a complete list of tissue positive controls) appear brighter due to the greater amount of starting RNA in comparison to single PSC colonies. Ngn3 was not expressed at detectable levels in individual PSC colonies. However Ngn3 mRNA was detected in a sample of 5 pooled (P) PSC colonies, suggesting that it is present in differentiated PSC colonies but perhaps at low levels. E. Single cells from dissociated PSC colonies co-express PDX-1 (red) and nestin (green) by immunostaining. Note that the nucleus in this fluorescence micrograph is labeled with both DAPI (blue) and PDX-1, giving it a pink appearance. The white arrows indicate double-positive cells.

FIG. 2. PSC colonies generate all 3 major neural cell lineages. A-B. When individual PSC colonies were differentiated, they were found to generate β₃-tubulin⁺ neurons (red), occasionally forming large neuronal networks as shown in B. Scale bars are 50 μm for A, 200 μm for B. C-E. β₃-tubulin⁺ neurons that were generated by PSCs (C) co-expressed the more mature neuronal marker MAP2 (green) (D and overlay, E), thus confirming their neuronal identity. Scale bars, 50 μm. F-G. PSCs generated GFAP⁺ astrocytes (green). Scale bars, 20 μm. H. O4⁺ oligodendrocytes also were generated by PSC colonies (green). Scale bar, 20 μm. All nuclei were counterstained with DAPI (blue) for purposes of quantification. Refer to Table 1 for relative proportions of each neural cell type produced by PSCs. I. RT-PCR analyses confirm the presence of mRNA for neuronal and glial makers. Individual differentiated clonal PSC colonies all expressed detectable levels of β₃-tubulin and MAP2, but not GFAP. However, GFAP mRNA was detected in a sample of 5 pooled (P) PSC colonies, suggesting that it is present in differentiated PSC colonies but at lower levels. This is in accordance with the relatively lower percentages of glial than neuronal progeny determined by immunocytochemistry (Table I). Only single colony RNA isolates that were found to express β-actin were considered. Note that positive control (+) bands appear brighter due to the greater amount of starting RNA in comparison to single PSC colonies.

FIG. 3. Progeny from two distinct embryonic primary germ layers are generated by single, clonally-derived PSCs that are present in pancreatic islet and ductal cell isolates. A-B. Single islet (A) and duct (B) PSC colonies generated both β₃-tubulin⁺ neurons (red) and insulin⁺ or C-peptide⁺ β-cells (green). Note that although only one combination of β₃-tubulin and insulin or C-peptide is shown for each of islet and ductal PSC colonies, both islet and ductal PSC colonies contained insulin⁺ and C-peptide⁺ cells in combination with β₃-tubulin. The white arrows indicate insulin⁺ and C-peptide⁺ cells. Scale bars, 50 μm. C-D. To confirm that the insulin⁺ cells represented true β-cells and were generating insulin protein de novo, colonies were co-labeled with antibodies against PDX-1 and C-peptide (C) or insulin (D). These micrographs illustrate single colonies with cells positive for both PDX-1 (red) and C-peptide or insulin (green). Scale bars, 25 μm. E-F. Insulin⁺ cells (red) all co-express C-peptide (green) as illustrated by the merged field (yellow) (E) and C-peptide⁺ cells (green) all co-express Glut2 (red) as shown in the merged field (yellow) (F). Scale bars, 50 μm. Although only one example of each is illustrated, both islet- and ductal-derived PSC colony progeny exhibited these patterns. In all micrographs nuclei have been counterstained with DAPI for purposes of quantification. Note that in C and D nuclei appear pink due to the co-localization of DAPI and PDX-1. Refer to Table 1 for the proportion of β-cells produced by single PSCs. G. RT-PCR analyses confirm that single clonal differentiated PSC colonies express many characteristic islet/β-cell markers, showing that PSCs generate true β-cells de novo in culture. Only single colony RNA isolates that were found to express β-actin were considered. Note that positive control (+) bands appear brighter due to the greater amount of starting RNA in comparison to single PSC colonies.

FIG. 4. Insulin⁺ cells generated de novo from PSCs demonstrate glucose-stimulated Ca2⁺ responses and glucose-stimulated insulin release. A-B. Bright field and fluorescence micrographs demonstrating YFP⁺ cells from AdRIP2EYFP-infected islet—(A) and ductal—(B) derived PSC colonies. C-D. Calcium traces for islet—(C) and ductal—(D) derived PSC colonies demonstrating glucose-stimulated [Ca²⁺]ᵢ responses, which were augmented by the addition of either GLP-1 or TEA, respectively. The addition of the voltage-dependent Ca²⁺ channel blocker verapamil (VER) returned the [Ca²⁺]ᵢ to basal levels. Shown above the Ca²⁺ trace are fluorescence micrographs of YFP⁺ cells and the ratiometric Fura images (pseudocoloured according to the scale shown to the right) corresponding to the numbered time points on the trace. Note that in (C), the YFP⁻ cell does not demonstrate a glucose response. These Ca²⁺ traces are representative of at least 5 independent experiments. E-F. Demonstration of increased insulin release by islet—(E) and ductal—(F) derived PSC colonies in response to high glucose (HG) alone or with the addition of GLP-1, TEA, or to Carbachol (Carb) alone. The addition of verapamil (VER) abolished the glucose-stimulated insulin release. These data were generated from 34 independent experiments.

FIG. 5. PSC colonies generate multiple islet endocrine subtypes and exocrine cells. A. When individual PSC colonies were differentiated, they were found to generate glucagon⁺ α-cells (green) and somatostatin⁺ δ-cells (red). Cells co-expressing these hormones were never observed. Note that this field depicts only a portion of a larger differentiated PSC colony. The arrangement of endocrine cells in these colonies is suggestive of either multiple divisions of one local progenitor cell within the colony, or that there may be a type of “community effect” whereby endocrine cells of similar phenotype tend to differentiate in close contact with each other. B. PSC colonies generated cells characteristic of the exocrine compartment of the pancreas, amylase⁺ acinar cells. C-D. A large proportion of the cells generated by individual clonal PSC colonies were large, flat cells with characteristic morphology and arrangement that expressed SMA (C) and nestin (D), typical of pancreatic stellate cells. All nuclei were counterstained with DAPI (blue) for purposes of quantification. Refer to Table 1 for relative proportions of each pancreatic cell type produced by PSCs. Scale bars, 25 μm.

FIG. 6. PSCs are not general endodermal or mesodermal precursors, nor are they ES-like stem cells or neural crest precursors. A. Individual PSC colonies were assayed by RT-PCR for the presence of the early endoderm markers GATA-4 and HNF3β. None of the colonies tested expressed either marker, suggesting that PSCs are not generalized endodermal precursors. B. mRNA for Oct4 and Nanog, genes characteristic of ES cells, was not detected in any of the single clonal PSC colonies assayed, showing that PSCs are not ES-like pluripotent stem cells. C. Brachyury and GATA-1, markers of mesodermal tissue, were not detected by RT-PCR in PSC colonies, showing that PSCs are not of mesodermal origin. D. Clonal PSC colonies do not exhibit a characteristic neural crest progenitor profile. Although PSC colonies do express Slug and Snail, and a proportion of them express detectable levels of p75, they do not express many other characteristic neural crest markers including Pax3, Twist, Sox10, or Wnt1 by RT-PCR analysis. Only single colony RNA isolates that were found to express β-actin were considered. Note that positive control (+) bands appear brighter due to the greater amount of starting RNA in comparison to single PMP colonies.

FIG. 7. PSCs are present in both nestin⁺ and nestin⁻ cell fractions from both islet and ductal cell isolates, but all PSC colonies are nestin⁺ after 7 days in vitro. A. Some PSC cells are nestin⁺ at the outset of culture; 100% of PSC colonies from these cultures express nestin. B. PSC colonies also arise from cells in the nestin⁻ cell fraction; 100% of the resultant colonies 7 days later are nestin⁺. The left-side pictures are light micrographs and right-side pictures are fluorescent images of pancreas cultures from nestin-GFP transgenic tissue. Left and right pairs represent images of the same field. These findings are consistent with the RT-PCR analysis that also indicated that PSC colonies express nestin. C. Immunostaining confirms the presence of nestin protein (green) in the cells of undifferentiated acutely dissociated PSC colonies. Nuclei have been counterstained with DAPI (blue).

FIG. 8. Differentiated PSC colonies contain β-cells that co-express Pax6 (red) and C-peptide (green). The left panel illustrates all DAPI-stained (blue) nuclei present in the same field of view. Note that this figure depicts only a portion of a larger differentiated PSC colony.

### DETAILED DESCRIPTION OF THE INVENTION

The invention relates to the first clonal isolation of pancreatic stem cells (PSC) and multipotential progenitor (PMP) cells from the pancreas. The cells were obtained from adult pancreas, but are also available from child (non-adult) or embryonic pancreas tissue. These stem cells proliferate and form floating clonal cell colonies. Such pancreas colonies arise from stem cells present in both islet- and duct-derived populations, and from both nestin⁺ and nestin⁻ cell fractions. Intriguingly, clonal pancreas colonies express markers characteristic of both neural and pancreatic precursors. Upon differentiation, clonal pancreas colonies generate multiple types of neural progeny including mature neurons. Surprisingly, pancreas colonies generate a significantly higher proportion of neurons than do adult brain-derived clonal neurospheres. In addition, pancreas colonies generate islet endocrine cell types including mature pancreatic insulin-producing β-cells, glucagon-producing α-cells, and somatostatin-producing δ-cells. The de novo generated β-cells are functional in that they exhibit glucose-dependent Ca²⁺ responsiveness and insulin release. Pancreas colonies also generate acinar cells characteristic of the exocrine pancreas and pancreatic stellate cells, demonstrating that these unique precursor cells are multipotential not only for multiple pancreatic cell types but also for both neuroectodermal and endodermal cell types. This is the first report of a robust adult somatic cell population from the pancreas that is capable of reliably and reproducibly generating clonal progeny characteristic of both endocrine and exocrine pancreatic lineages, and indeed progeny characteristic of more than one primary germ layer.

There have been a number of other striking studies suggestive of multi-germ layer lineage potential of adult bone marrow cells⁵¹, neural stem cells⁵² and perinatal inner ear cells¹¹, but to date such cells have not been isolated from the adult pancreas. PSCs represent the first clonally characterized adult somatic cell type from the pancreas capable of reliably and reproducibly generating progeny characteristic of more than one embryonic primary germ layer.

The culture period utilized in the present study was much shorter than what has reportedly caused transformation events in adult mouse cells in vitro. Further, transformation events manifest themselves differently in different cell isolates⁵³. In the present study, for the consistently observed neurons and various islet endocrine cells to be the result of a transformation, the identical transformation event would have had to occur in every one of the more than 100 clonal pancreas colonies assayed from more than 14 separate experiments. This shows that the unusual combination of differentiated cell types generated by pancreatic precursors was not the result of transformation events.

PSCs were present in both nestin⁺ and nestin⁻ cell fractions. Other studies have similarly suggested that nestin expression is not related to pancreatic progenitor identity^{54,55,56}. However, PSC colonies derived from both initially nestin⁺ and nestin⁻ stem cells ultimately exhibited nestin expression in the majority of cells, suggesting that nestin may be expressed at least transiently by progenitors downstream from the colony-initiating PSC. PSCs may be a different nestin⁺ stem cell than those found in vivo to represent pancreatic epithelial cell progenitors^{57,58} or endothelial cells⁵⁹. Moreover, in differentiated PSC colonies, nestin expression was associated with pancreatic stellate (mesenchymal) cells, as has been previously described^{24,60}.

The relationship between PSCs and other types of previously described adult precursors also was investigated. PSCs represent an entirely novel type of intrinsic adult pancreatic precursor cell, and are the first pancreatic cells to be identified at the single-cell level as being capable of generating multiple pancreatic and neural cell types. As such, PSCs represent a source of cells for replacement strategies.

This invention discloses the isolation of a stem cell from both adult mouse islet- and duct-derived tissue as well as adult human islet- and duct-derived tissue. Similar stem cells are optionally obtained from adult human islet- and duct-derived tissue and from mouse and human islet- and duct-derived tissue. This is the first isolation and proof that a pancreatic stem cell capable of generating both multiple neural and pancreatic cell types is present in the adult mammalian islet- and duct-derived tissue.

(A) Cells of the Invention:

The present inventors have clonally isolated novel stem cells from the adult murine pancreas. The term “clonal” is used herein to mean that the cells are derived from a single isolated cell. The unique application of a serum-free colony-forming assay to pancreatic cells enabled the identification of a subpopulation of stem cells from each of pancreatic islet and ductal populations. These cells can proliferate in vitro to form clonal colonies that co-express neural and pancreatic precursor markers. Upon differentiation, individual clonal colonies produce distinct populations of neurons and glial cells; pancreatic endocrine β-, α-, and δ-cells cells; pancreatic exocrine cells and pancreatic stellate cells. Moreover, the de novo generated β-cells demonstrate glucose-dependent Ca²⁺-responsiveness and insulin release. Pancreas colonies do not express markers of ES cells, nor genes suggestive of mesodermal or neural crest origins. These cells represent a novel adult intrinsic pancreatic stem cell population and represent a source for cell-based therapeutic strategies.

Accordingly, the invention provides for stem cells which are clonally isolated and purified from the pancreas. The pancreas stem cells (PSC) are optionally from any animal, preferably mammal, more preferably human or mouse. Adult, child or embryonic pancreatic tissue is useful to obtain PSC cells. In another embodiment, the invention provides for pancreatic cells produced from the PSC cells. The pancreatic cells optionally include alpha cells, delta cells, beta cells, pancreatic exocrine cells and pancreatic stellate cells. In another embodiment, the de novo generated β-cells demonstrate glucose-dependent Ca²⁺-responsiveness and insulin release. In yet another embodiment, the invention provides for neural cells produced from the stem cells. The neural cells optionally include neurons, glial cells and oligodendrocytes.

The pancreatic stem cells may be transformed or transfected with a heterologous gene. For example, heterologous expression of the transcription factors PDX-1, Neurogenin-3, or Pax4 is useful. Genes are identified to drive differentiation towards the pancreatic beta cell lineage or neural cell lineage. One of skill in the art would understand that there are a myriad of genes which may be effective dependent on the desired outcome. The growth or differentiation of the pancreatic stem cells is optionally stimulated by a growth factor. Proliferation is readily induced by administering genetically engineered cells which secrete growth factors into the pancreas. In one embodiment, the growth factor is epidermal growth factor and/or fibroblast growth factor. In another embodiment, self-renewal and proliferation are enhanced by the presence of BIO ((2′Z,3′E)-6-Bromoindirumbin-3′oxime). In another embodiment, application of Wnt pathway activators, e.g. recombinant Wnt ligand proteins, or transfection/infection of Wnt pathway modulator genes are employed.

The invention also includes an isolated colony or sphere (a colony may be a sphere or another form of colony if cultured on a surface) comprising PSC cells and/or cells derived therefrom. The invention also includes an isolated PSC cell expressing one or more cell markers and/or one or more neural-specific mRNA molecules, as described herein. The PSC cells of the invention are multipotent and have multilineage potential.

Methods of Isolating and Culturing PSC Cells

One stimulates the pancreatic stem cells to proliferate and differentiate to achieve and replace the compromised parts of the pancreas or nervous system. As a result of this invention, the stem cells are optionally cultured in vitro to generate large numbers of new stem or progenitor cells. The stem cells may also be differentiated to provide a source of healthy differentiated pancreatic or neural cells.

Accordingly, it is an object of the invention to provide a method for isolating pancreatic stem cells from the pancreas of a mammal by obtaining pancreatic tissue from the mammal, dissociating the tissue into single cells, culturing the cells, and isolating cells which proliferate to form floating clonally-derived pancreatic precursor cell colonies. For example, cells are optionally cultured for at least 7 to 14 days in order to effectively discriminate the highly-proliferative (colony-forming) cells. Optionally the method further includes differentiating the cells that proliferated into pancreatic cells or neural cells such as those described in this application.

Appropriate culture conditions are readily determined for the robust self-renewal of PSC cells. One strategy to obtain self-renewing divisions of PSCs is overexpression of Notch, as Notch signaling has been demonstrated to be critical for preventing the differentiation and promoting the self-renewal of other cell types^{49,50}. The invention includes the cells of the invention overexpressing Notch. Another signaling molecule that has been implicated in regulation of stem cell self-renewal is Wnt (Paxianos et al. “The elements of stem cell self-renewal: a genetic perspective”, Biotechniques. 2003 December; 35(6): 124.0-7). Cells transformed with Notch or Wnt are useful in methods for isolating pancreatic stem cells from the pancreas of a mammal as described in this application. BIO is a highly potent, selective, reversible inhibitor of the GSK-3α/β protein kinases. The GSK-3α/β protein kinases have a critical role within the Wnt-signalling pathway, with their inhibition by BIO leading to an activation of the Wnt pathway. The Wnt pathway is involved with the specification/development of multiple tissues. Accordingly, the invention also provides for a method of passaging PSC cells, comprising contacting the PSC cells with BIO to passage the PSC cells. Preferably the method includes passaging the PSC cells through at least: 5, 10, 20 or 50 passages. One embodiment includes culturing clonally derived primary PSC spheres in serum-free media in the presence of BIO, EGF and FGF, and passaging the cells in serum-free media in the presence of BIO, EGF and FGF.

The invention also provides for a method of passaging PSC cells, comprising contacting the cells with a surface to permit the cells to attach to the surface and produce a monolayer of cells then passaging the cells. One embodiment involves, contacting PSC spheres with the surface and developing an outgrowth of cells on the surface to form a monolayer of cells, followed by passaging the cells in the spheres and the cells in the outgrowth. Optionally the method includes culturing clonally derived primary PSC spheres, plating them on a gelatin substrate in the presence of media comprising EGF, FGF and a low percentage of fetal calf serum, for example 1%, allowing the spheres to attach and an outgrowth of cells to develop, passaging the spheres and outgrowth in the media conditions as an adherent monolayer. Cells are optionally frozen and stored in liquid nitrogen.

In another embodiment, the invention provides a method of culturing beta cells comprising clonally isolating pancreatic stem cells from the pancreas of a mammal and differentiating the pancreatic stem cells into beta cells, wherein the beta cells secrete insulin in response to glucose.

(B) Therapeutic Applications of the Invention

Use of Cells of the Invention:

The invention includes the use of the cells of the invention for treatment of diseases and disorders of the nervous system and pancreas as described herein.

Accordingly, the present invention provides for the use of PSC cells or cells derived therefrom for the treatment of diseases and disorders of the nervous system and pancreas. The invention also provides for the preparation of a medicament comprising the cells of the invention for the treatment of diseases and disorders of the nervous system and pancreas. The invention also relates to a method of treating a disease, disorder or abnormal state of the pancreas or nervous system in a subject in need thereof comprising administering cells of the invention to the subject, preferably by implanting the cells in the subject.

The diseases that are treated by cells of the invention are optionally selected from the group consisting of neural damage or trauma, neural paralysis (eg. spinal cord injury), Alzheimer's disease, Parkinson's disease, Creutzfelt-Jacob disease, pancreatic degeneration, diabetes, pancreatitis, and cancers of the pancreas.

The invention further relates to a method of treating a disease, disorder or abnormal state of the pancreas or nervous system by administering a proliferative agent to a subject to stimulate proliferation of PSC cells so that the PSC cells produce pancreatic cells in the pancreas. Preferably, administration of the proliferative agent is targeted either specifically to the pancreas or specifically to the nervous system. Selective delivery of an agent may be accomplished by site-specific injection, implantation of agent-delivery devices, or targeted expression of “proliferative agent” genes to specific cell/tissue types. According to one embodiment of this invention, the proliferative agent is a growth factor. The proliferative agent is optionally contacted with pancreatic stem cells in a subject. Accordingly, the invention also includes a method of stimulating proliferation of pancreatic progenitor cells, by contacting the cells with growth factors (for example, EGF or FGF2). Accordingly, another aspect of the invention is a method of treating a disease, disorder or abnormal state of the pancreas or nervous system, by stimulating proliferation of pancreatic stem cells in a subject. The method of treatment is useful in treating a disease, disorder or abnormal physical state of the pancreas such as one selected from the group consisting of type I or type II diabetes, pancreatitis, pancreatic degeneration and cancers of the pancreas The cells are also useful for treating any neural or pancreatic disease or disorder that would benefit from cell transplant. An individual suffering from a degenerative disease, disorder or abnormal physical state of the pancreas or nervous system may also be treated by implanting the PSC cells or cells derived from the pancreatic stem cells into the individual. Implantation of cells is optionally carried out by injecting cells into the individual, such as by implanting the cells in any tissue capable of supporting the pancreatic cells as live cells secreting insulin in response to glucose. For example, cells are optionally implanted in the pancreas or the liver. In the treatment of diabetes, implantation of a beta-cell containing graft in multiple locations can have a beneficial effect as the graft becomes vascularized, i.e it “sees” the blood and blood glucose levels, and then can respond by releasing appropriate insulin into the blood, thus normalizing aberrant blood glucose levels. Thus, various implantation locations yield benefits to blood glucose. For other diseases, implantation into the site of the damaged tissue is preferable for functional restoration.

It has been previously demonstrated that transplantation of beta cells/islets provides therapy for patients with diabetes (Shapiro et al., 2000). The shortage in islet cells has, prior to this invention, represented a limitation for large-scale use of islet transplantation to cure patients with diabetes. PSC cells are an alternative source which provide enough islet cells to prevent or treat diabetes. As well, beta-cell progenitors (such as PSC cells) provide a source of cells for autologous cell transplant that eliminates the need for immunosuppressive regimens that themselves result in significant morbidity. (Shapiro, A M., Lakey, J R., Ryan, E A., Korbutt, G S., Toth, E., Warnock, G L et al. Islet transplantation in seven paitents with type I diabetes mellitus using a glucocorticoid-free immunosuppressive regimen. New England Journal of Medicine 343, 230-238 (2000).) Accordingly, the cells are usefully transplanted in order to prevent or treat the occurrence of diabetes. Several strategies are useful for transplantation. For example, isolation and purification of the stem cells themselves, followed by transplantation, or transplantation of whole PSC colonies, or transplantation of beta cells purifed from the PSC colonies.

Accordingly, the PSC cells are useful as sources of transplantable tissue or cells. PSC cells can be removed from a donor and transplanted into a recipient either before or after differentiation into pancreatic cells. In one embodiment, the donor and the recipient are the same individual which is an autologous transplant. This invention satisfies the needs outlined above in that the PSC cells of this invention (1) are accepted by the patient because they can be taken from the patient's own body, (2) are safe in that the patient is not receiving cells or tissue from another source, (3) are effective in that the PSC cells can be differentiated into pancreatic cells for implantation and survive during and after implantation, and (4) offer the potential to provide long term relief of the symptoms of conditions associated with loss of one or more pancreatic cell types.

Accordingly, in one embodiment of the invention, where the mammal is a human and is suffering from a disease, disorder or abnormal physical state of the pancreas, the method includes implanting the PSC cells or pancreatic cells differentiated from the PSC cells, into the pancreas of the human. Where the mammal is a human and is not suffering, from a disease, disorder or abnormal physical state of the pancreas, the method includes implanting the PSC cells or pancreatic cells differentiated from the PSC cells into a second human who is suffering from the disease, disorder or abnormal physical state. These approaches are adapted for use with the nervous system by implanting PSC cells or nerve cells differentiated from the PSC cells.

The invention also comprises transplantation of pancreatic stem cells or cells derived therefrom into a subject and differentiating the cells into mature pancreatic and neural cells. Stem cells or cells derived therefrom are optionally transplanted into recipient mice and other mammals. The cells integrate into the mammals, for example, the beta cells produce insulin without immune rejection or abnormal cell development. The materials and methods employed for transplantation will be readily apparent to those skilled in the art of cellular transplantation and are further described herein.

The stem cells of the invention, and/or cells derived therefrom are optionally transplanted in diabetic subjects (eg. cells are optionally implanted in or on the pancreas, the liver, the kidney or other organs or tissues capable of supporting the cells) to secrete glucose and treat diabetes. In one embodiment, the invention provides for a method of treating diabetes or other disease of the pancreas described herein, comprising differentiating PSC cells into beta cells and administering the beta cells to a subject, wherein the beta cells secrete insulin in response to glucose.

The invention also relates to the use of the cells of this invention to introduce recombinant proteins into the diseased or damaged pancreas or nervous system. The cells act as a vector to transport a recombinant molecule, for example, or to transport a sense or antisense sequence of a nucleic acid molecule. In the case of a recombinant molecule, the molecule would contain suitable transcriptional or translational regulatory elements.

Suitable regulatory elements may be derived from a variety of sources, and they may be readily selected by one of ordinary skill in the art. Examples of regulatory elements include: a transcriptional promoter and enhancer or RNA polymerase binding sequence, a ribosomal binding sequence, including a translation initiation signal. Additionally, depending on the vector employed, other genetic elements, such as selectable markers, may be incorporated into the recombinant molecule. The recombinant molecule may be introduced into stem cells or pancreatic or neural cells differentiated from stem cells of a patient using in vitro delivery vehicles such as retroviral vectors, adenoviral vectors, DNA virus vectors, amplicons and liposomes. They may also be introduced into these cells using physical techniques such as microinjection and electroporation or chemical methods such as coprecipitation and incorporation of DNA into liposomes.

Suitable regulatory elements may be derived from a variety of sources, and they may be readily selected by one of ordinary skill in the art. If one were to upregulate the expression of the gene, one would insert the sense sequence and the appropriate promoter into the vehicle. If one were to downregulate the expression of the gene, one would insert the antisense sequence and the appropriate promoter into the vehicle. These techniques are known to those skilled in the art.

Pharmaceutical Compositions

It is another object of the invention to provide a pharmaceutical composition for use in implant therapy comprising the cells of the invention in a pharmaceutically acceptable carrier, auxiliary or excipient. The invention therefore provides a composition (preferably a pharmaceutical composition) comprising clonal cells and/or cells derived therefrom. Preferably, the composition comprises (or consists of, or consists essentially of) clonal cells and/or cells derived therefrom and is free of non-clonal cells which are regarded as impurities. The pharmaceutical compositions of this invention are useful to treat patients having degenerative diseases, disorders or abnormal physical states of the pancreas and typically include an acceptable carrier, auxiliary or excipient.

On this basis, the pharmaceutical compositions optionally include stem cells or pancreatic cells or neural cells, in association with one or more pharmaceutically acceptable vehicles or diluents, and contained in buffered solutions with a suitable pH and isoosmotic with the physiological fluids. The methods of combining growth factor or cells with the vehicles or combining them with diluents is well known to those skilled in the art. The composition optionally includes a targeting agent for the transport of the active compound or cells to specified sites within the pancreas or nervous system, such as specific cells, tissues or organs.

The pharmaceutical compositions are readily prepared by known methods for the preparation of pharmaceutically acceptable compositions which are then administered to patients, and such that an effective quantity of the cells is combined in a mixture with a pharmaceutically acceptable vehicle. Suitable vehicles are described, for example in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The cells of the invention are formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation. Dosages to be administered depend on patient needs, on the desired effect and on the chosen route of administration. The compositions can be fortopical, parenteral, local, intrahepatic or intrapancreatic use. One of skill in the art would understand that the site of administration would depend on the desired outcome/treatment. For example, in the treatment of diabetes, administration of beta-cells in multiple locations can have a beneficial effect as they become vascularized, and then can respond by releasing appropriate insulin into the blood, thus normalizing aberrant blood glucose levels. Thus, various administration sites may yield benefits to blood glucose. For other diseases, administration into the site of the damaged tissue maybe preferable for functional restoration. If treatment of nerve degeneration is desired, any location of nerve damage would be an injectable delivery site.

The pharmaceutical compositions optionally include PSC cells genetically engineered to produce an active compound or substance, in association with one or more pharmaceutically acceptable vehicles or diluents, and contained in buffered solutions with a suitable pH and iso-osmotic with the physiological fluids. The active compound or substance can be a recombinant protein or a recombinant molecule described herein. The methods of combining cells with the vehicles or combining them with diluents is well known to those skilled in the art. The composition could include a targeting agent for the transport of the active compound to specified sites within the pancreas, such as specific cells, tissues or organs.

Screening Assays of the Invention:

Examples of screening assays are provided below.

Toxicity Testing

PSC cell cultures also provide useful assay cultures for toxicity testing. Toxicity testing is done by culturing stem cells or cells differentiated from stem cells in a suitable medium and introducing a substance, such as a pharmaceutical or chemical, to the culture. The stem cells or differentiated cells are examined to determine if the substance has had an adverse effect on the culture. Accordingly, the invention provides a method for identifying a substance which is toxic to PSC cells or pancreatic cells or neural cells differentiated from PSC cells comprising introducing the substance to a PSC cell culture or a pancreatic or neural cell culture differentiated from a PSC culture, and determining whether the cell culture is adversely affected by the presence of the substance. In one embodiment, the cell culture is adversely affected if at least half of the cells die.

Drug Development Testing:

Drug development testing may be done by developing derivative cell lines, for example a pathogenic pancreatic cell line, which may be used to test the efficacy of new drugs. Affinity assays for new drugs may also be developed from the stem cells, differentiated cells or cell lines derived from the stem cells or differentiated cells. The cells of the invention may also be used in a method for identifying a pharmaceutical which is useful to treat a disease, disorder or abnormal state of the pancreas or nervous system. Accordingly, the present invention provides a method for identifying a pharmaceutical useful in the treatment of a disease, disorder or abnormal state of the pancreas or nervous system comprising introducing the pharmaceutical to a PSC cell culture or a pancreatic or neural cell culture differentiated from a PSC cell culture, and determining whether the culture is affected by the presence of the pharmaceutical, for example, the culture may be affected (i.e. compared to a control culture) and, as a result, have increased cell division, increased cell differentiation or secrete increased insulin in response to glucose.

In one embodiment, the invention provides a method for identifying a pharmaceutical useful in the treatment of diabetes or other disease of the pancreas described herein comprising introducing the pharmaceutical to a cell culture comprising beta cells differentiated from PSC cells, contacting the beta cells with glucose and determining whether insulin secretion increases or decreases.

Modulators of the Invention:

There has been much effort to isolate progenitor cells and stem cells and determine which peptide growth factors, hormones and other metabolites influence stem cell renewal and production of stem cells, which conditions control and influence the differentiation of progenitor cells into specialized tissue cells, and which conditions cause a stem cell to develop into a particular type of cell. Accordingly, the cells also provide a culture system from which genes, proteins and other metabolites involved in cell development can be isolated and identified. The composition of cells may be compared with that of differentiated cells in order to determine the mechanisms and compounds which stimulate production of stem cells or mature cells.

In one embodiment, the invention also includes a method for screening for modulators of pancreatic stem cell differentiation comprising:
a. culturing pancreatic stem cells under conditions that produce differentiation in the absence of the modulator;
b. detecting any differentiation of the cells and cell types generated, if any, in the presence of the modulator compared to differentiation and cell types generated in the absence of the modulator;
c. determining whether the modulator affects the differentiation of the cells.

The modulators optionally comprise any culturing conditions that may modulate cellular differentiation. The invention also includes a method for screening for differentiation factors of cellular development comprising:
a. culturing pancreatic stem cells in the presence of the differentiation factor;
b. allowing cells to differentiate;
c. detecting differentiation of the cells, if any.

The method optionally further comprises determining whether the differentiation of the cells comprises pancreatic cell or neural cell development. The invention also includes a method for screening for differentiation factors of cellular development comprising:
a. culturing the pancreatic stem cells in the presence of the differentiation factor.
b. detecting any differentiation of the cells.

The method optionally further comprises determining whether the cells differentiate into a homogenous uniform cell base, for example a neural cell base or pancreatic cell base. The cells of the invention are optionally cultured in a transplantation media.

Kits of the Invention:

Another object of the invention is to provide a kit, containing at least one type of cell selected from a group consisting of the pancreatic progenitor cells and the pancreatic cells or neural cells and directions for use in treatment of disease or disorders. The cells are preferably in a pharmaceutical composition. The kit may be used for the treatment of a disease, disorder or abnormal physical state of the pancreas or nervous system.

### EXAMPLES

The present inventors have demonstrated the isolation and characterization of a novel pancreas-derived multipotential precursor cell. These PSCs are present at low frequency (˜0.02-0.03%) throughout the pancreas, in both nestin⁺ and nestin⁻ cell fractions from both islet and ductal isolates. Single PSCs are capable of proliferation and colony formation in vitro, as determined by mixing experiments of marked and unmarked cells and more definitively by single cell analyses. PSC colonies express both neural and pancreatic precursor markers, and generate all three types of neural progeny (neurons, astrocytes, and oligodendrocytes), in addition to three islet endocrine subtypes, mature β-cells, α-cells, and δ-cells, as well as exocrine acinar cells and pancreatic stellate cells. Moreover, the new β-cells were shown to be functional through the demonstration of glucose-stimulated [Ca²⁺]ᵢ response, glucose-stimulated insulin release (and augmentation of this release in response to GLP-1 and TEA), and insulin release in response to carbachol.

The invention will be illustrated by the results discussed below which are provided as examples and do not limit the scope of the invention.

### Example 1

### Pancreas Colonies Arise Clonally from Single Islet and Ductal Cells

To show cells isolated from adult pancreatic islets and ductal tissue would proliferate in vitro, we utilized defined serum-free media conditions that are typical for the isolation of brain-derived neural stem cells, but which have not been applied to cultures of pancreatic cells. In these conditions, neural stem cells clonally proliferate to form floating cell colonies called neurospheres⁶. Pancreatic islets and ductal tissue were separately dissociated into single cells and plated at low density in the serum-free medium containing epidermal growth factor (EGF) and fibroblast growth factor 2 (FGF2).

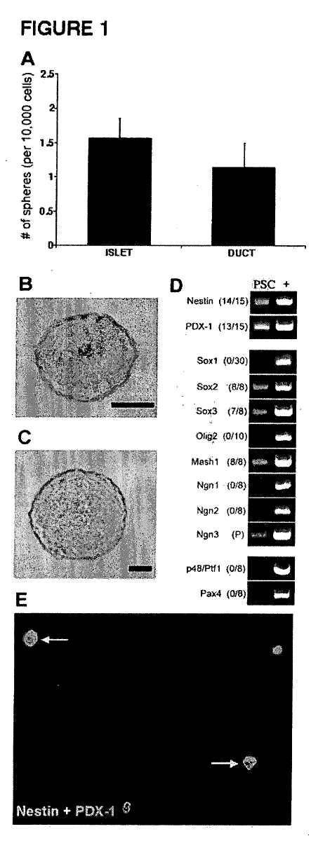

By 7 days in vitro, floating colonies which morphologically resembled neurospheres had formed in both islet and duct cultures (FIG. 1). There was no significant difference in the number of colonies formed from islet (1/6410 cells) and ductal (1/8850 cells) cells (p>0.05) (FIG. 1A). Indeed, throughout the following analyses there were no differences noted between islet- and ductal-derived progenitor colonies, henceforth they will be referred to collectively as pancreas colonies or (based on the following analyses) PSC colonies. Although the PSC colonies were morphologically similar to neurospheres (FIG. 1C), on average they were smaller in diameter (104±8.6 μm PSC colony compared to 263±7.7 μm neurosphere⁷ (Seaberg & van der Kooy, 2002) (FIG. 1B) and did not increase significantly in size upon lengthening of the culture period. Each PSC colony contains 2000-10,000 cells.

A number of experiments were performed to confirm that the PSC colonies were arising due to the proliferation of individual cells and not due to cellular aggregation. First, mixing experiments were conducted in which equal proportions of wildtype (white, unmarked) and marked (GFP⁺) cells from animals constitutively expressing GFP⁸ were dissociated and plated together at a final density of 20 cells μl⁻¹, and the resulting colonies were assayed for the number of white colonies, green colonies, and mixed colonies. Mixed colonies are indicative of cellular aggregation. This type of analysis has been used successfully for demonstrating the in vitro clonal derivation of other precursor colonies, including brain-derived neural stem cells⁹, retinal stem cells¹⁰ and inner ear stem cells¹¹. We found that of the 114 PSC colonies assayed, some were wholly unmarked, some were apparently wholly GFP⁺, and 0/114 were mixed. These data indicate that PSC colonies do not arise by aggregation when pancreas cells are plated at 20 cells μl⁻¹ or lower, but rather by the proliferation of single (either unmarked or GFP⁺) pancreatic precursors.

To confirm the clonality of PSC colonies more rigorously, single cell analyses were performed. Cells were plated at a density of 0.05 cells μl⁻¹ in 96-well plates.

At the outset of the culture period, wells were assayed for the presence of single cells, and only wells containing single cells were included in the further analysis. A total of 15,335 single cells were followed in these analyses, of which 5 (0.03%) formed colonies. This percentage of colony-forming cells is similar to the observed ˜0.02% (FIG. 1A) of cells that form colonies in routine culture conditions of 20 cells μl⁻¹ (a density 400-fold greater than that used for the single cell analysis). Indeed, colonies formed at a slightly higher frequency from single cell per well cultures than from cultures of 20 cells μl⁻¹, indicating that there may be a subtle inhibitory influence from neighbouring cells. These data show that the PSC colonies arise from the clonal proliferation of single cells and not from cellular aggregation. Thus, all subsequent analyses were performed on clonal PSC colonies that were generated from the proliferation of a single cell.

### Example 2

### Single Pancreas Colonies Express Both Neural and Pancreatic Precursor Markers

The pancreatic transcription factor PDX-1 is necessary for pancreatic development and is one of the earliest genes expressed in the developing^{12,13} and regenerating¹⁴ pancreas. Indeed, PDX-1⁺ cells generate both exocrine and endocrine compartments of the pancreas during development¹³. Other markers of pancreatic progenitors include p48/Ptf1, Pax4, and Ngn3¹⁵. Ngn3 is also expressed in neural precursors, as are Ngn1 and Ngn2¹⁶, Nestin⁵, Sox1-3¹⁷, Mash-1⁸, and Olig2¹⁹. To determine whether PSCs expressed markers more characteristic of neural or pancreatic precursors, RT-PCR analysis was performed on single colonies for PDX-1, p48/Ptf1, Pax4, Ngn1-3, Nestin, Mash-1, Sox1-3, and Olig2. Nearly all PSC colonies tested expressed both nestin (14/15) and PDX-1 (13/15) (FIG. 1D; see also Supplementary Table I for comparison with neurospheres). As well, single cells from acutely dissociated PSC colonies co-expressed PDX-1 and Nestin (an example is shown in FIG. 1E). Individual PSC colonies also expressed Sox2, Sox3, Mash-1, and Ngn3 (but not Pax4, p48/Ptf1, Olig2, Sox1, Ngn1, and Ngn2). This showed that individual clonal PSC colonies expressed markers characteristic of both pancreatic and neural precursors and hinted that the PSC cell might be a novel stem cell that could generate both neural and pancreatic progeny.

### Example 3

### Individual Pancreatic Progenitors Generate Multiple Neural Lineages

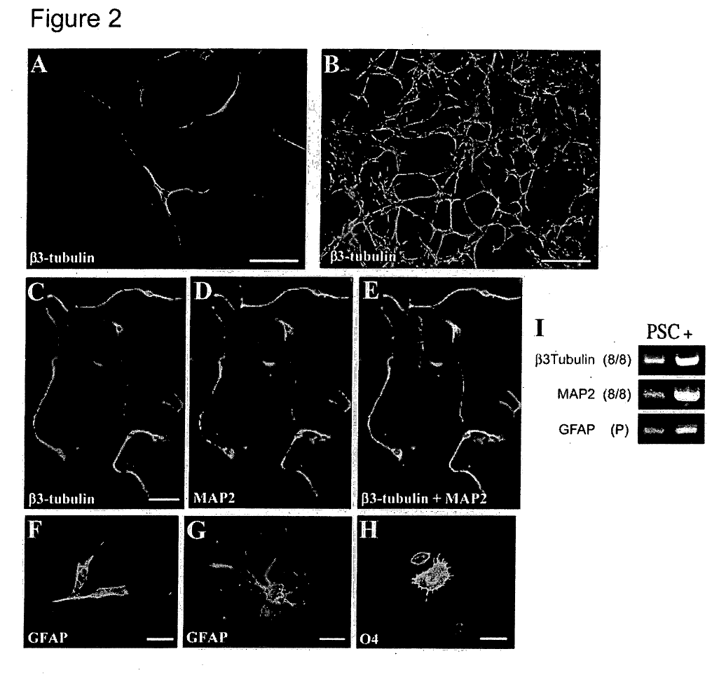

To show that PSC colonies would generate progeny characteristic of neural or pancreatic cell lineages, individual clonal PSC colonies were removed from mitogen-containing media, replated on an adherent substrate and allowed to differentiate for 7 days. PSC colonies from both islet and duct cultures generated β₃-tubulin⁺ neurons, GFAP⁺ astrocytes and O4⁺ oligodendrocytes (FIG. 2A, F, G, H). These are the same cell types routinely generated by brain-derived neural stem cells when neurospheres are differentiated in this manner. However, under identical differentiation conditions, the PSC colonies generated very different proportions of these cell types compared to brain-derived neurospheres (Table I). For example, brain-derived neurospheres generate a much higher proportion of GFAP⁺ astrocytes (84.2±1.4%) than PSC colonies (7.4±1.3%), although neurospheres and PSC colonies generated similar numbers of O4⁺ oligodendrocytes (4.3±1.7% and 2.4±0.7%, respectively). Notably, PSC colonies generated a significantly greater proportion of neurons (26.4±3.8%) than did brain-derived neurospheres (3.7±0.6%). In rare cases, differentiated PSCs formed colonies consisting primarily of neurons with extensive networks of neuronal processes (FIG. 2B). Neurons also were co-labeled with antibodies against MAP2 (FIG. 2C-E), a later neuronal marker that, along with the observed characteristic neuronal morphology, confirms that these cells are indeed mature neurons. The detection of β₃-tubulin and MAP2 protein was critical because islet cells in certain types of monolayer culture can extend short neurite-like processes, and thus neural cells cannot be identified on the basis of morphology alone. However it warrants comment in these studies that cells immunopositive for any endocrine or exocrine pancreatic marker were never found to extend neurites. In addition to the morphological and immunocytochemical evidence, the presence of mature neural lineages in the clonal differentiated PSC cultures was confirmed by RT-PCR of individual colonies for β₃-tubulin and MAP2 (FIG. 2I). GFAP was undetectable in single colonies but was detected when 5 colonies were pooled together, suggesting that it is present in differentiated PSC colonies but at lower levels. This is in accordance with the relative percentages of glial and neuronal progeny determined by immunocytochemistry (Table 1).

### Example 4

### In Addition to Neural Cell Types, Pancreatic Stem Cells Generate β-Cells

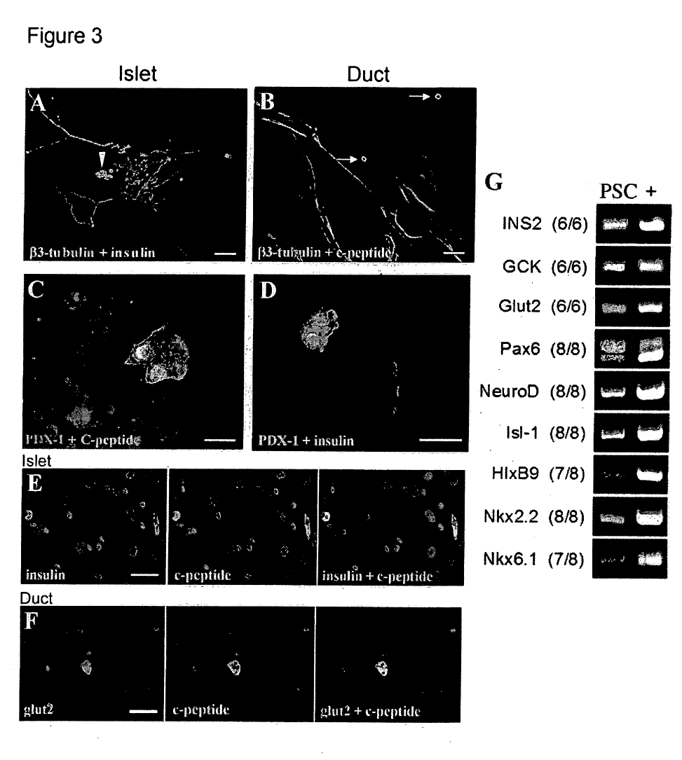

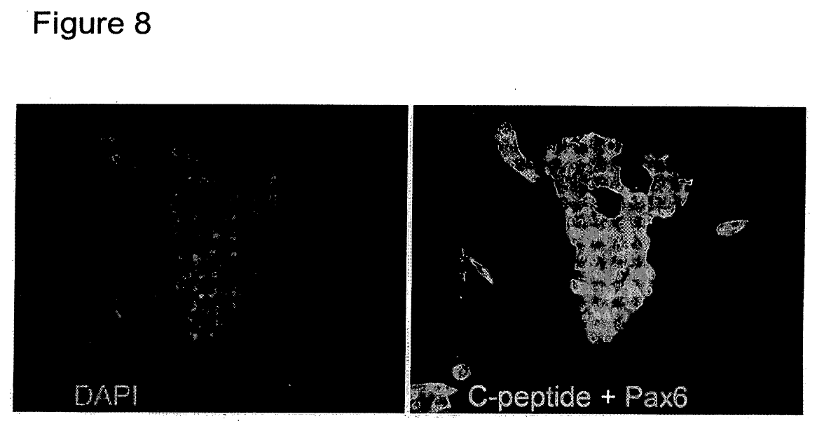

Surprisingly, the same clonal PSC colonies that generated neural progeny also generated insulin⁺ and C-peptide⁺ pancreatic β-cells (n=100 individual clonal PSC colonies). This result was found for both islet (FIG. 3A) and ductal (FIG. 3B) clonal colonies by the identification of β₃-tubulin⁺ and either insulin⁺ or C-peptide⁺ cells within the same single clonal colony, indicating that the original colony-forming PSC cells were multipotential for both neural and pancreatic lineages. We confirmed that the insulin⁺ cells identified in differentiated PSC cultures represented bona fide mature β-cells and we ruled out the possibility that an unrelated cell type was simply concentrating insulin from the culture medium²⁰. This was the rationale for the utilization of antibodies against C-peptide, a cleavage product of the insulin pro-hormone that is released during insulin production. To further confirm that the insulin⁺ cells were β-cells, a series of double-labeling experiments were performed. Single cells from differentiated PSC colonies co-expressed insulin or C-peptide and PDX-1, a transcription factor expressed by mature β-cells¹³ (FIG. 3C, D). All single C-peptide⁺ cells co-expressed PDX-1. Further, single cells were co-labeled with C-peptide and insulin (FIG. 3E) or C-peptide and Glut2 (FIG. 3F). Every single insulin⁺ cell co-expressed C-peptide⁺, and every C-peptide⁺ cell co-expressed Glut2, demonstrating that insulin immunoreactivity is not a consequence of uptake from the culture media. Co-labeled cells expressing both C-peptide and Pax6 were also found in these cultures (FIG. 8). Moreover, all of these immunocytochemical results were found for both islet and ductal-derived PSC colonies, although only one example of each is shown in the relevant figures.

To show that differentiated PSC colonies expressed other characteristic markers of β-cells/islet cells, RT-PCR was performed on single clonal differentiated colonies for Insulin II (Ins2), Glucokinase (GCK), Glut2, Pax6, Beta2/NeuroD, Hlxb9, Isl-1, Nkx2.2, and Nk6.1. Single differentiated PSC colonies contained mRNA corresponding to all of these markers (FIG. 3G). Taken together, these data show that PSCs generate de novo mature α-cells upon differentiation.

### Example 5

### β-Cells Generated De Novo from PSCs Exhibit Glucose-Dependent Ca²⁺ Responsiveness and Insulin Release

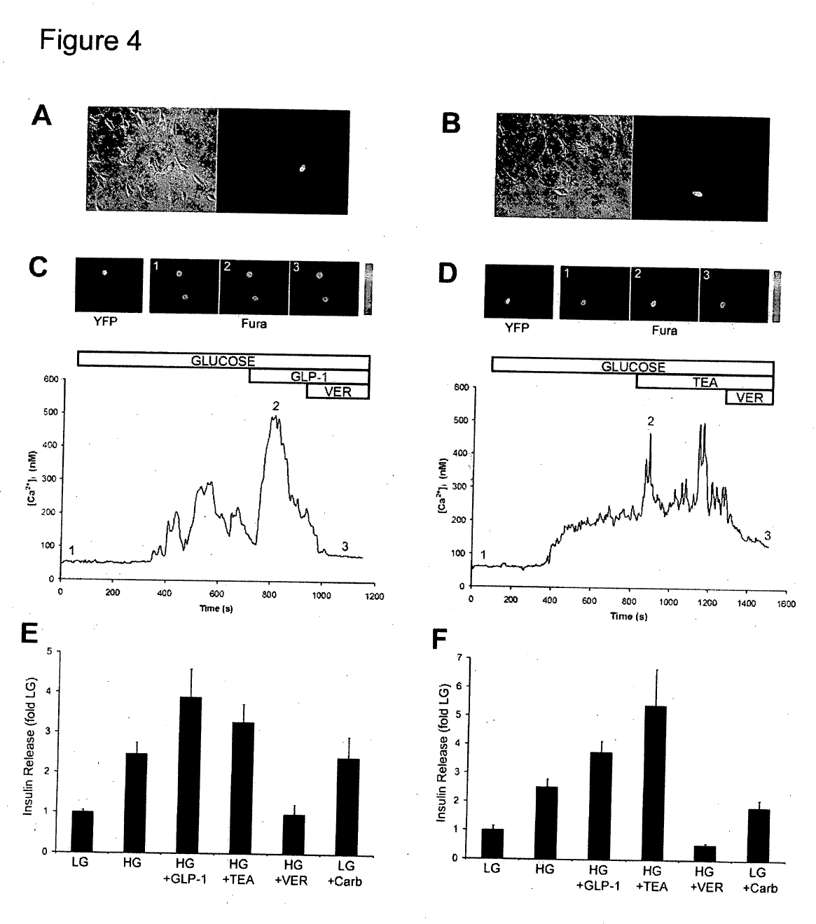

The multiple β-cell markers demonstrated by both RT-PCR and immunocytochemical co-labeling studies provide strong evidence for the presence of β-cells in single clonal differentiated PSC cultures. These cells are also capable of normal β-cell function. To show that these cells function as β-cells, intracellular Ca²⁺ ([Ca²⁺]ᵢ) imaging studies were performed. Single cells were identified for [Ca²⁺]ᵢ imaging by prior infection with AdRIP2EYFP, an adenovirus in which the expression of enhanced yellow fluorescent protein (EYFP) was placed under the control of the rat insulin II gene promoter (RIP2). YFP expression has been demonstrated to be insulin⁺ β-cell specific in whole islets of Langerhans (Kang et al., 2003). The fact that YFP⁺ cells were identified in these differentiated PSC cultures also shows that these cells are in fact β-cells (FIG. 4A, B). YFP⁺ cells from both islet- and ductal-derived PSC cultures exhibited a [Ca²⁺]ᵢ response to stimulation by glucose (FIG. 4C, D). This response was augmented by the addition of the physiological secretagogue glucagon-like peptide-1 (GLP-1), which is known to stimulate β-cells in a glucose-dependent manner²¹, or tetra ethyl ammonium (TEA), a compound which inhibits delayed rectifier K⁺ currents and potentiates the glucose-stimulated insulin response²². GLP-1 and TEA produced similar responses in both islet- and ductal-derived PSC progeny, although only one example is depicted for each in the [Ca²⁺]ᵢ traces shown in FIG. 4. Further, this [Ca²⁺]ᵢ response was abolished by the addition of the voltage-dependent Ca²⁺ channel blocker verapamil. These results show that the YFP⁺ cells present in cultures of differentiated PSC colonies are glucose-responsive. Further, when insulin release is measured directly by radioimmunoassay, PSC-derived cells clearly demonstrate increased insulin secretion in response to glucose alone or to glucose +GLP-1 or +TEA (FIG. 4E, F). These cells also secrete insulin in response to carbachol, a cholinergic agonist that is capable of stimulating insulin release even under low glucose conditions²³. In contrast, verapamil abolishes glucose-stimulated insulin release to basal levels. These data clearly show that there are cells present in cultures of differentiated PSC colonies (from both islet and ductal cell fractions) that exhibit the functional properties of β-cells.

### Example 6

### Pancreatic Stem Cells Clonally Generate Multiple Islet and Pancreatic Cell Types

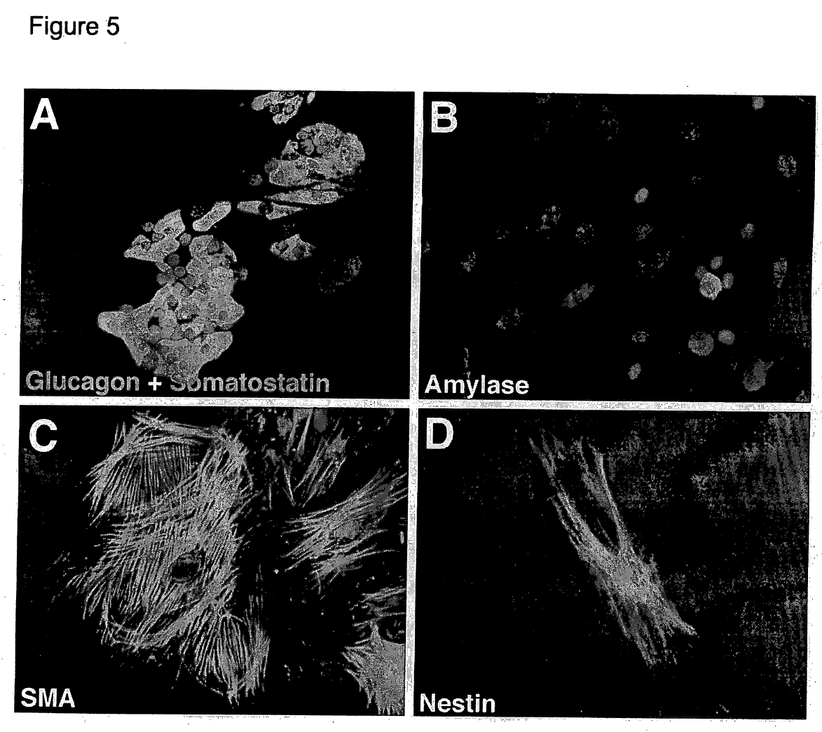

To show that PSCs could generate other subtypes of pancreatic islet endocrine cells, differentiated clonal PSC colonies were tested for the presence of α-cells and δ-cells using antibodies specific for glucagon and somatostatin, respectively. Interestingly, we found that both α-cells (6.3±2.0%) and 8-cells (4.5±0.6%) were generated by clonal PSC colonies (FIG. 5A, Table I). Insulin⁺ cells were also found in the same PSC colonies that generated these other endocrine cell types. Importantly, glucagon, somatostatin, and insulin defined non-overlapping cell populations, showing that these cells represent differentiated endocrine subtypes.

To show that PSCs represent a more general pancreatic precursor capable of generating cells characteristic of the exocrine compartment of the pancreas, differentiated clonal PMP colonies are tested for the presence of acinar (exocrine) cells. Colonies were stained with antibodies against amylase, which marks pancreatic exocrine acinar cells. PSCs did reliably generate amylase⁺ acinar cells (6.2±1.2%) (FIG. 5B, Table I), showing that PSCs are common stem cells for both exocrine and endocrine lineages of the pancreas. One of skill in the art could also readily determine whether the cells produce pancreatic ductal epithelial cells.

PSCs clearly generate neuroectodermal cells. One of skill in the art could also readily determine whether the cells produce other non-neural ectodermal derivatives. One of skill in the art could also readily determine whether the cells produce another endodermal cell type, hepatocytes. One of skill in the art could also readily determine whether the cells are generalized endodermal precursors.

We accounted for up to 50% of the differentiated progeny of individual clonal PSC colonies. The remaining cells were large and flat, with large nuclei, and were usually found in a characteristic sheet-like arrangement. In contrast, the neural cells and particularly the pancreatic endocrine cells were much smaller. To show the phenotype of the many large, flat cells generated by differentiated PSC colonies, antibodies against nestin and smooth muscle actin (SMA) were employed. We found that many large, flat cells generated by PSC colonies expressed nestin (49.6±2.9% of total DAPI⁺ nuclei) and SMA (57.4%±7.0% of total DAPI⁺ nuclei) (FIG. 5C, D; Table I). Because nestin and SMA were expressed in an overlapping cell population with a common morphological phenotype, these cells represent pancreatic stellate cells, which have been shown to display this characteristic morphology and also to express nestin and SMA²⁴.

### Example 7

### Self-Renewal of PSCs

In order to show the capacity of pancreatic precursors for self-renewal, individual clonal colonies were dissociated into single cells and replated in the same mitogen-containing media conditions used for the isolation of primary colonies, and then assayed after 7-14 days in vitro for the presence of secondary colonies. Some (<1%) primary PSC colonies generated small secondary colonies, suggesting that pancreatic precursors did not undergo many self-renewing divisions in these culture conditions. In these particular conditions, PSC cells did not undergo many self-renewing divisions in vitro. Cell viability was high after colony dissociation, and indeed after 7-14 days in vitro many single viable cells remained in the culture wells. One of skill in the art would readily be able to adjust culture conditions to increase formation of secondary colonies and obtain benefits of the cells acting as a stem cell or restricted progenitor²⁵. Cells were cultured using known techniques and it was identified that they act as stem cells (stem cells are defined by two properties: their multipotentiality and long-term self-renewal capacity²⁵).

Two examples of strategies that have shown pancreatic precursor self-renewal and population expansion are provided below.

1) Using the standard PSC sphere colony formation assay, the (commercially available) compound, BIO ((2_Z,3_E)-6-Bromoindirubin-3_-oxime) has been included. BIO is a highly potent, selective, reversible inhibitor of the GSK-3α/β protein kinases. The GSK-3α/β protein kinases have a critical role within the Wnt-signalling pathway, with their inhibition by BIO leading to an activation of the Wnt pathway. The Wnt pathway is involved with the specification/development of multiple tissues. With BIO inclusion during the primary serum-free sphere formation and subsequent serum-free passaging, PSC spheres can be passaged indefinitely. PSC spheres have been successfully passaged, currently up to the 9th passage over a period of 5 months. The number of sphere-forming cells and resultant clonal spheres expanded (over 30-fold) during this period.

2) Proliferation of the adult and embryonic pancreatic stem cell is optionally stimulated in a chemically defined serum-free medium in the presence of growth factors. The cells respond to growth factors such as fibroblast growth factor (FGF2), epidermal growth factor (EGF). A strategy had been the use of monolayer culturing. In this method, clonally derived primary PSC spheres are plated on a gelatin substrate in the presence of the growth factors, EGF and FGF, and 1% fetal calf serum. The spheres attach and an outgrowth of cells develops. The spheres and outgrowth are successively passaged in the above media conditions as an adherent monolayer and can be passaged indefinitely. This monolayer of pancreatic stem cells/precursors has been passaged 10 times and expanded over a period of 5 months. This cell population could be successfully frozen/stored in liquid nitrogen and thawed. This method starts with the primary clonal spheres (derived from a single cell) before changing to monolayer culture.

Both of these techniques demonstrate the self-renewal/expansionary capacity of our initial clonal PSC population.

### Example 8

### PSCs Exist in Both Nestin⁺ and Nestin⁻ Pancreatic Cell Fractions

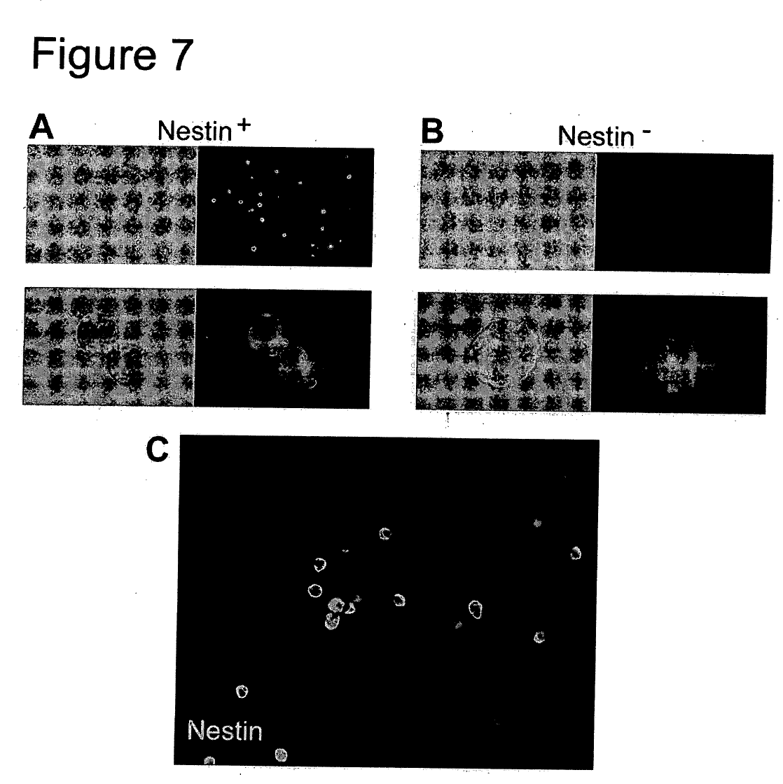

Although PSC colonies expressed nestin as determined by RT-PCR analysis, this result does not resolve the issue of whether the colony-initiating cells are nestin⁺. To determine whether PSCs are nestin⁺ cells, a transgenic mouse model in which enhanced GFP is expressed under the control of the nestin second-intron enhancer²⁶ was utilized. Islet and ductal cells were analyzed for GFP expression by FACS analysis, sorted into nestin⁺ (FIG. 7A) and nestin-fractions (FIG. 7B) and cultured. Approximately 5% of islet cells and 1% of ductal cells were nestin⁺. However, the nestin⁺ subpopulation was not enriched for PSC colony-forming cells. Indeed, 1/4286 nestin⁺ cells yielded colonies and 1/2514 nestin⁻ cells formed colonies, suggesting that the nestin⁺ cells were in fact slightly depleted in the number of PSC colony-forming cells. These FACS results were confirmed with a second independent nestin-GFP transgenic mouse line²⁷, strongly suggesting that nestin expression is not able to predict PSC identity.

Interestingly, although colonies formed from both nestin⁺ and nestin⁻ cells, all of the colonies assayed at the end of the culture period were nestin⁺ (FIG. 7A, B). The transgene expression was confirmed by independent experiments to detect endogenous nestin protein by immunocytochemistry (FIG. 7C). Thus, consistent with the finding that PSC colonies are nestin⁺ according to RT-PCR analysis (FIG. 1D), even the PSCs contained within the nestin⁻ fraction of cells (or their progeny within the colony) acquire nestin expression at some point during proliferation and colony formation. These nestin⁺ cells did not demonstrate co-expression of CD31 or E-cadherin by immunocytochemistry suggesting that the nestin⁺ cells present in undifferentiated PSC colonies are not endothelial or epithelial cells, respectively.

### Example 9

### Pluripotent ES-Like Cells

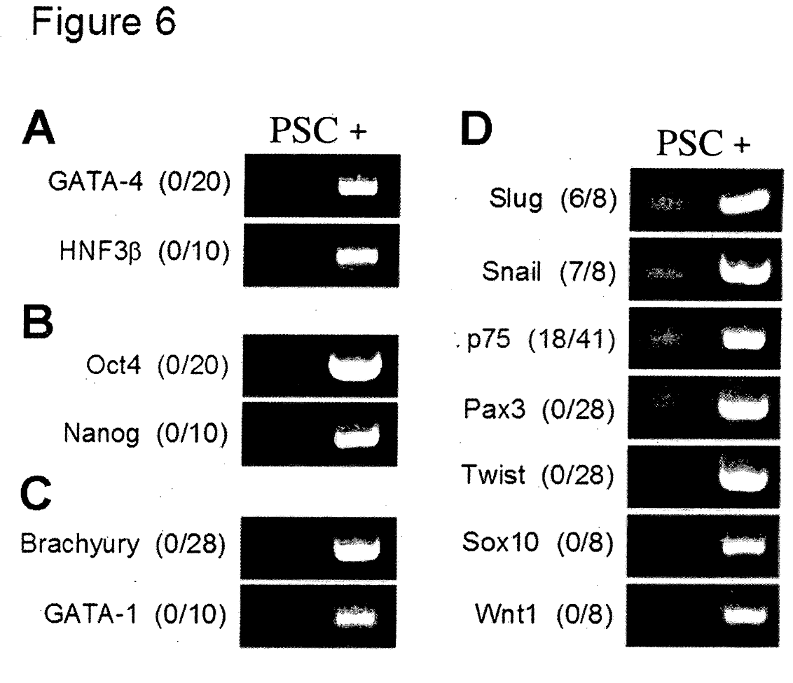

Cell sorting based on nestin expression did not enrich for the pancreatic colony-forming precursors, so several other candidate markers were investigated. It has been suggested that a small number of the pluripotent stem cells present in the inner cell mass of the pre-implantation embryo (capable of generating all embryonic lineages, including germ cells) might never differentiate, but instead may persist and seed adult tissues. Further, it has been hypothesized that these rare pluripotent cells may be responsible for the numerous recent observations of unexpected adult somatic tissue plasticity²⁸. Oct4 is a transcription factor critical to the development of totipotent cells²⁹. In order to determine whether multipotential pancreas colonies were arising from a population of Oct4⁺ pluripotent stem cells resident in the adult pancreas, a transgenic mouse expressing enhanced GFP behind the Oct4 promoter was utilized. Although there was a very small number of GFP⁺ cells in both islet (0.4%) and duct (0.6%) isolates, none of these GFP⁺ cells formed colonies. Because in the adult mouse Oct4 expression is thought to be restricted to germ cells³⁰, the presence of GFP⁺ cells in the adult pancreas of these transgenic mice was surprising. To pursue this finding by determining if Oct4 or Nanog, which is also expressed in ES cells³¹ were transcribed in pancreatic precursors, primary islet cells and single PSC colonies were analyzed by RT-PCR for Oct4 mRNA. All of the clonal colony samples tested were negative for Oct4 and Nanog expression (FIG. 6B). Similarly, primary islet cells did not express detectable levels of Oct4 or Nanog mRNA by RT-PCR, suggesting that Oct4-GFP transgene expression in pancreas cells may represent very low levels of Oct4 or ectopic expression from the transgene. Taken together, these results indicate that PSCs do not correspond to a population of putative pluripotent ES-like stem cells in adult tissues.

### Example 10

### Mesodermal Origin

The suggestion has been made that a primitive mesodermal stem cell originating from the bone marrow exists in multiple adult tissues, and may adopt tissue-specific characteristics depending on the local environment³². Stem cell antigen 1 (Sca-1) is a cell surface protein of the Ly-6 gene family expressed by bone marrow-derived hematopoietic stem cells³³. In an effort to determine whether the colony-forming PSCs were Sca-1⁺ and thus related to primitive mesodermal stem cells, islet and ductal cells were marked with a Sca-1 antibody and sorted by FACS analysis. Although 9% of islet cells and 15% of ductal cells were Sca1⁺, none of the Sca1⁺ cells formed pancreatic colonies. To confirm that PSCs are not mesodermal in origin, single colony RT-PCR was performed for mesoderm markers Brachyury and GATA-1. None of the clonal colony samples tested were positive for Brachyury or GATA-1 mRNA (FIG. 6C). In addition, differentiated PSC colonies were analysed with immunocytochemistry for expression of MyoD, a marker of mesoderm-derived myoblasts and differentiated skeletal muscle cells³⁴. There were no MyoD⁺ cells found in differentiated PSC colonies. Thus, PSCs are neither Sca1⁺, GATA-1⁺, nor Brachyury⁺, and they do not generate typical mesodermal progeny, suggesting that they do not represent a primitive mesodermal precursor or one that is derived from bone marrow.

### Example 11

### Neural Crest Cells

Nestin-positive precursor cells that can produce neurons in vitro have been isolated from adult skin (skin-derived precursors or SKPs)³⁵, and may represent a neural crest derivative³⁶. Because pancreatic precursors are a similarly unusual source of neurons, pancreas colonies were assayed for the expression of neural crest markers by RT-PCR.

Clonal PSC colonies do not express the neural crest markers Pax3³⁷ or Twist³⁸ (FIG. 6D). These markers are expressed by the aforementioned SKPs (McKenzie et al., 2003). Similarly, clonal PSC colonies do not express neural crest markers Sox10³⁹ or Wnt1⁴⁰ (FIG. 6D). PSC colonies did express Slug and Snail⁴¹ and less than half of the pancreas colonies assayed expressed detectable levels of p75 neurotrophin receptor mRNA (FIG. 6D), which is expressed in neural crest stem cells⁴². However, p75 is not specific to neural crest stem cells or their derivatives but also is expressed in forebrain neurons⁴³, embryonic islets⁴⁴ and in the present study, brain-derived neurospheres (Table 2). PSCs also do not exhibit characteristics of mesodermal cells, in contrast to other precursors that may have a neural crest origin³⁶. Although expression of Slug and Snail are detected, PSCs do not express the full cluster of markers that have been found co-expressed in neural crest stem cells or progenitors derived from neural crest³⁶. Taken together, these data suggest that PSCs do not express a typical neural crest progenitor profile and are not neural crest derivatives.

### Example 12

### Human Pancreatic Stem Cells

We have obtained human pancreatic tissue samples and determined that they contained pancreatic precursors by placing them in our standard serum-free plus growth factor conditions. Results show that the human tissue contains a sub-population of proliferative cells, forming colonies akin to those described from mouse tissue.

Methods

Animals, Cell Isolation and Culture

The mice used in these studies included 6-week old male Oct4-EGFP animals that express enhanced GFP under the control of the Oct4 promoter, nestin-EGFP animals which express enhanced GFP under the control of the nestin second-intron enhancer³⁶, GFP animals which constitutively express GFP in all cells (Jackson), and wildtype BalbC animals (Charles River). Islets were isolated by collagenase digestion of the pancreas and Ficoll density gradient centrifugation. After centrifugation islets were handpicked for further purification⁶². Ductal tissue was similarly handpicked to ensure purity.

Isolated islets and ductal tissue were then incubated with trypsin (Sigma) at 37° C. and triturated with a small-borehole siliconized pipette into a single cell suspension. Viable cells were counted using Trypan Blue (Sigma) exclusion and plated at 20 cells μl⁻¹ or less in defined serum-free medium (SFM)⁶³ containing B27 (Gibco-BRL), 10 ng ml⁻¹ FGF2 (Sigma), 2 μg ml⁻¹ heparin (Sigma), and 20 ng ml⁻¹ EGF (Sigma) for 7-14 DIV. For some experiments, the following growth factors were added 100 pM hepatocyte growth factor (Sigma), 10 ng ml⁻¹ keratinocyte growth factor (Calbiochem), 10 ng ml⁻¹ insulin-like growth factor-1 (Upstate Biotech), 2 nmol L⁻¹ Activin-A (Sigma), 10 mM nicotinamide (Sigma), and 10 nM exendin-4 (Sigma). For clonal analysis, primary cells were diluted to a density of 0.05 cells μl⁻¹ and plated in Nunclon 96-well plates (Nalge Nunc International). Each well was scored after plating for the presence of a single cell. Only wells that contained single cells at the outset of the culture period were subsequently assayed for colony formation. For differentiation, whole individual pancreas colonies were removed from the aforementioned mitogen-containing media and transferred to wells coated with MATRIGEL basement membrane matrix (15.1 mg ml⁻¹ stock diluted 1:25 in SFM, Becton-Dickinson) in SFM containing 1% FBS without dissociation. As the colony differentiates, cells migrate out of the spherical colony to form a flat monolayer. To ensure accurate assay of the progeny from single pancreatic precursors, each well contained only a single clonal pancreas colony. Neurospheres were generated from adult mice for comparison purposes as described previously⁷.

FACS Analysis

Islet and ductal cells were isolated as described, and cells were sorted with an EPICS Elite Cell Sorter (Beckman-Coulter). In the case of Nestin-eGFP and Oct4-eGFP transgenic cells, separate single cell suspensions of islet and ductal cells were sorted into separate fractions based on GFP fluorescence. For the Sca-1 sorting experiment, cells were first labeled with PE-Sca-1 mouse monoclonal (1:250; Pharmingen), and sorted into separate cell fractions based on PE fluorescence.

Immunocytochemistry, Cell Quantification and Statistical Analysis

Fixation and immunocytochemical analysis of pancreas colonies was performed as described previously for neurospheres⁷. See Supplementary Methods for a list of the primary and secondary antibodies used, as well as positive control tissues for each antibody. For cell quantification, the numbers of neurons, astrocytes, oligodendrocytes, β-cells, α-cells, δ-cells, acinar cells, stellate cells, stellate/neural precursor cells were determined by counting the numbers of 3-tubulin⁺, GFAP⁺, O4⁺, insulin⁺ cells, glucagon⁺, somatostatin⁺, amylase⁺, SMA⁺, and nestin⁺ cells respectively, as a percentage of DAPI⁺ nuclei in at least 3 photographed fields of differentiated cells per colony (n≧10 colonies). The absolute number of cells counted per cell type to determine the percentages (Table I) ranged between 2000-4000 cells each. Statistical analyses consisted of Student's t-tests. A p value of <0.05 was considered to represent a significant difference between groups.

RT-PCR Analysis

Total RNA was extracted from individual colonies using an RNeasy extraction kit (Qiagen). Reverse transcription and PCR were carried out using a OneStep RT-PCR kit (Qiagen) in a GeneAmp PCR System 9700 (Applied Biosystems) according to kit instructions. PCR reactions were performed for 35-40 cycles due to the relatively small amount of starting material involved in single-colony RT-PCR. It is important to note that it is difficult to draw conclusions about mRNA quantity from these methods. All samples were treated with DNAse to avoid contamination with genomic DNA. Controls run without reverse transcriptase did not produce bands. Forward and reverse primers (5′-3′), expected product size, annealing temperatures and positive control tissues are as described herein. Only single colony RNA isolates that were found to express β-actin were considered for further analysis. If β-actin was found in a single colony RNA isolate but the gene of interest was not, 5 colonies were pooled and re-assayed. When expression was found in pooled but not single samples, this result was interpreted as mRNA presence in PSC colonies, but perhaps at low levels.

RIP-YFP Adenovirus and [Ca²⁺]ᵢ Imaging Studies

An adenovirus in which the expression of enhanced yellow fluorescent protein (EYFP) was placed under the control of the rat insulin 11 gene promoter (RIP2) (AdRIP2EYFP) was constructed as described⁶⁴. Expression of EYFP has been demonstrated to be restricted to infected insulin⁺ β-cells in whole islets of Langerhans⁶⁴. PSC colonies were infected with AdRIP2EYFP for 48 hours from day 5-7 of differentiation. Colonies were trypsinized, dissociated and re-plated on laminin/polyornithine-coated glass coverslips for 24 hours in RPMI-1640 media containing 5 mM glucose, 10% FCS, and 10 mM HEPES prior to imaging. Experiments were performed in a KRB solution consisting of (in mM): 129 NaCl, 4.8 KCl, 5 NaHCO₃, 2.5 CaCl₂, 1.2 MgSO₄, 1.2 KH₂PO₄, 10 HEPES and 0.1% BSA. Individual RIP-YFP⁺ cells were visualized and Ca²⁺ imaging using Fura2 was performed on these single cells as previously described⁶⁵.

Insulin Release Studies

PSC colonies were pooled and differentiated (8 per well, 96-well Matrigel-coated plates) for 7 days as described. Twenty-four hours prior to secretion studies, the medium was changed to supplemented RPMI-1640 medium as outlined above. Differentiated PSC colonies were pre-incubated in low glucose (2.5 mM) KRB solution (LG-KRB) for 1 hour. The solution was changed to 150 μl of fresh LG-KRB and the cultures were incubated for 1.5 hours to establish the basal level of insulin release. Cultures were incubated for a further 1.5 hours in either LG-KRB alone or with experimental agents (20 mM glucose, 30 nM GLP-1, 10 mM TEA, 100 μM verapamil or 100 μM carbachol). Insulin was measured using an RIA kit (Linco). Insulin release during the experimental 1.5 hour incubation was compared to the level determined during the basal 1.5 hour incubation period for each individual well to obtain a percent change. The data are expressed relative to the percent change measured for the LG-KRB to LG-KRB alone condition.

Supplementary Methods:

Immunocytochemistry

Primary antibodies utilized included anti-tubulin isotype III mouse monoclonal (IgG) (1:500, Sigma), anti-microtubule associated protein 2 (MAP2) rabbit polyclonal (IgG) (1:200; Chemicon), anti-glial fibrillary acidic protein (GFAP) rabbit polyclonal (IgG) (1:400; BTI), anti-GFAP mouse monoclonal (IgG) (1:400; Sigma), anti-O4 mouse monoclonal (IgM) (1:100; Chemicon), anti-PDX-1 rabbit polyclonal (IgG) (1:750; Chemicon), anti-insulin guinea pig polyclonal (IgG) (1:1000; Linco), anti-insulin mouse monoclonal (IgG) (1:500; Sigma), anti-C-peptide guinea pig polyclonal (IgG) (1:1000; Linco), anti-nestin mouse monoclonal (IgG) (1:1000; Chemicon), anti-nestin rabbit polyclonal (IgG) (1:2000; kind gift from Dr. R. McKay), anti-amylase rabbit polyclonal (IgG) (1:100; Sigma), anti-somatostatin rabbit polyclonal (IgG) (1:200; ImmunoStar), anti-glucagon mouse monoclonal (IgG) (1:500; Sigma), anti-MyoD mouse monoclonal (IgG)(1:200; BD Pharmingen), anti-α-smooth muscle actin mouse monoclonal (IgG) (1:250; Sigma), anti-pan-cytokeratin (includes cytokeratins 1, 4, 5, 6, 8, 10, 13, 18, 19) mouse monoclonal (IgG) (1:100; Sigma), anti-fumarylacetoacetate hydrolase (FAH) chicken polyclonal (IgY) (1:10; kind gift from Dr. M. Grompe), anti-CD31 rat monoclonal (IgG) (1:500; Cymbus Biotechnology), anti-Glut2 rabbit polyclonal (IgG) (1:500; Chemicon), anti-E-cadherin rat monoclonal (IgG) (1:500, Sigma), and anti-Pax6 mouse monoclonal (1:500, Developmental Hybridoma Bank). Secondary antibodies were FITC goat anti-rabbit (1:200; Jackson), TRITC goat anti-rabbit (1:200; Jackson), TRITC goat anti-mouse (1:200; Jackson), FITC goat anti-mouse (1:200; Jackson), FITC donkey anti-rat (1:200; Jackson) and FITC donkey anti-guinea pig (1:200; Chemicon). Secondary-only wells were processed simultaneously using the identical protocol except that solutions did not contain primary antibodies. All secondary-only controls were negative for staining. Primary islet cells were used as a positive control for anti-PDX-I, anti-insulin, anti-glucagon, anti-somatostatin, anti-Glut2, and anti-C-peptide antibodies. Exocrine tissue was used as a positive control for anti-amylase, primary ductal cells for anti-pancytokeratin, primary gut tissue for anti-a-SMA, primary adult liver tissue for anti-FAH, liver vasculature for anti-CD31, undifferentiated ES cells for anti-E-cadherin, and retinal tissue for Pax6. Undifferentiated or differentiated adult forebrain neurospheres were used as positive control for anti-nestin, anti-O4, anti-β₃-tubulin, anti-MAP2, and anti-GFAP. Cell nuclei were counter-stained with DAPI. Fluorescent images were visualized and captured using an Olympus IX81 Motorized Inverted Research Microscope and captured using Olympus Microsuite Version 3.2 image analysis software (Soft Imaging System Corp.).

RT-PCR Analysis

Forward and reverse primers (5′-3′), expected product size, annealing temperatures were as follows: β-actin ATC ATG TTT GAG ACC TTC AA (SEQ ID NO:1) and TCT GCG CAA GTT AGG TTT TGT C (SEQ ID NO:2) (825 bp, 56° C.), β₃-tubulin CTC AGT CCT AGA TGT CGT GCG (SEQ ID NO:3) and GCG GAA GCA GAT GTC GTA GA (SEQ ID NO:4) (294 bp, 58° C.), Beta2/NeuroD CTG ATC TGG TCT CCT TCG TAC AG (SEQ ID NO:5) and GAT GCG AAT GGC TAT CGA AAG (SEQ ID NO:6) (540 bp, 58° C.), Brachyury AGT ATG AAC CTC GGA TTC AC (SEQ ID NO:7) and CCG GTT GTT ACA AGT CTC AG (SEQ ID NO:8) (857 bp, 56° C.), GATA-1 GGG AAG AGC AAC AAC ACG TTC (SEQ ID NO:9) and GTT TGC TGA CAA TCA TTC GCT (SEQ ID NO:10) (380 bp, 58° C.), GATA-4 AGC CTA CAT GGC CGA CGT GG (SEQ ID NO:11) and TCA GCC AGG ACC AGG CTG TT (SEQ ID NO:12) (809 bp, 58° C.), GFAP AGA ACA ACC TGG CTG CGT ATA G (SEQ ID NO:13) and TCT GCA AAC TTA GAC CGA TAC CA (SEQ ID NO:14) (290 bp, 58° C.), Glucokinase CAC CCA ACT GCG AAA TCA CC (SEQ ID NO:15) and CAT TTG TGG GGT GTG GAG TC (SEQ ID NO:16) (161 bp, 58° C.), Glut2 CCA CCC AGT TTA CAA GCT C (SEQ ID NO:17) and TGT AGG CAG TAC GGG TCC TC (SEQ ID NO:18) (325 bp, 58° C.), Hlbx9 ACA AGT ACC TGT CTC GAC CCA A (SEQ ID NO:19) and CTC AGA TGA GCA GTC GGA TGA (SEQ ID NO:20) (370 bp, 58° C.), HNF3β CAG ACC ACG CGA GTC CTA (SEQ ID NO:21) and CAT GAT CCA CTG ATA GAT CTC G (SEQ ID NO:22) (660 bp, 58° C.), Ins2 CCC TGC TGG CCC TGC TCT T (SEQ ID NO:23) and AGG TCT GAA GGT CAC CTG CT (SEQ ID NO:24) (212 bp, 58° C.), Isl-I TCT AAG GTG TAC CAC ATC GAG TGT (SEQ ID NO:25) and GCA GGC TGA TCT ATG TCG CTT (SEQ ID NO:26) (560 bp, 58° C.), MAP2 AAG GCC AAG AAC ACA CGA TTG (SEQ ID NO:27) and ACC AAG CCC TAA GCT TCG ACT M (SEQ ID NO:28) (650 bp, 58° C.), MashI AAG AAG ATG AGC AAG GTG GAG ACG (SEQ ID NO:29) and CAG AAC CAG TTG GTA AAG TCC AGC (SEQ ID NO:30) (257 bp, 55° C.), Nanog GCA CCA ACT CAA CTT CTG AGC (SEQ ID NO:31) and CTC GAG AGT AGC CAC CAT ATC (SEQ ID NO:32) (286 bp, 58° C.), Nestin ATA CAG GAC TCT GCT GGA GG (SEQ ID NO:33) and AGG ACA CCA GTA GAA CTG GG (SEQ ID NO:34) (410 bp, 56° C.), NgnI TGC ATC TCT GAT CTC GAC TGC (SEQ ID NO:35) and AGA TGT AGT TGT AGG CGA AGC G (SEQ ID NO:36) (406 bp, 58° C.), Ngn2 AAG CTC ACG AAG ATC GAG ACG (SEQ ID NO:37) and TGC CAG TAG TCC ACG TCT GAC (SEQ ID NO:38) (290 bp, 58° C.), Ngn3 CAT ACC TAG GGA CTG CTC CGA (SEQ ID NO:39) and CAT ACA AGC TGT GGT CCG CTA (SEQ ID NO:40) (320 bp, 58° C.), Nkx2.2 CTC TTC TCC AAA GCG CAG AC (SEQ ID NO:41) and AAC AAC CGT GGT AAG GAT CG (SEQ ID NO:42) (510 bp, 58° C.), Nkx6.1 TTC TCT GGA CAG CAA ATC TTC G (SEQ ID NO:43) and CTG AGT GAT TTT CTC GTC GTC A (SEQ ID NO:44) (300 bp, 58° C.), Oct4 GAA AGC AAC TCA GAG GGA AC (SEQ ID NO:45) and CTT CTC TAG CCC AAG CTG AT (SEQ ID NO:46) (497 bp, 56° C.), Olig2 GTG TCT AGT CGC CCA TCG TC (SEQ ID NO:47) and TCT TTC TTG GTG GAA GAC GTG (SEQ ID NO:48) (250 bp, 58° C.), p48/PtfI AGC CAC CAG CTA CAC GAA TAC T (SEQ ID NO:49) and GAG GAG GGA GAC CAT AAT CCG (SEQ ID NO:50) (635 bp, 58° C.), p75 GTG CGG GGT GGG CTC AGG ACT (SEQ ID NO:51) and CCA CAA GGC CCA CAA CCA CAG G (SEQ ID NO:52) (422 bp, 62° C.), Pax3 GGA GGC GGA TCT AGA AAG GAA GGA (SEQ ID NO:53) and CCC CCG GAA TGA GAT GGT TGA A (SEQ ID NO:54) (374 bp, 59° C.), Pax4 GTG AGC AAG ATC CTA GGA CGC (SEQ ID NO:55) and CGG GGA GAA GAT AGT CCG ATT (SEQ ID NO:56) (375 bp 58° C.) Pax6 TCA CAG CGG AGT GAA TCA GC (SEQ ID NO:57) and TAT CGT TGG TAC AGA CCC CCT C (SEQ ID NO:58) (377 bp, 58° C.), PDX-I GAC ATC TCC CCA TAC GAA GT (SEQ ID NO:59) and GTC CCG CTA CTA CGT TTC TTA T (SEQ ID NO:60) (451 bp, 56° C.), Slug TAC TGT ATG GAC ATC GTC GGC (SEQ ID NO:61) and ACA GTA CTT GCA GCT GAA CGA TT (SEQ ID NO:62) (330 bp, 58° C.), Snail AAG CCC AAC TAT AGC GAG CTG (SEQ ID NO:63) and AGT TGA AGA TCT TCC GCG ACT (SEQ ID NO:64) (420 bp, 58° C.), SoxI AAT CCC CTC TCA GAC GGT G (SEQ ID NO:65) and TTG ATG CAT TTT GGG GGT A (SEQ ID NO:66) (224 bp 58° C.), Sox2 GGA GTG GAA ACT TTT GTC CGA (SEQ ID NO:67) and TTC ATG TAG GTC TGC GAG CTG (SEQ ID NO:68) (420 bp, 58° C.), Sox3 AGA CTG AAC TCA AGA ACC CCG (SEQ ID NO:69) and GTC CTT CTT GAG CAG CGT CTT (SEQ ID NO:70) (435 bp, 58° C.), SoxI0 GGA GGT TGC TGA ACG AAA GTG (SEQ ID NO:71) and TCC ATG TTG GAC ATT ACC TCG (SEQ ID NO:72) (444 bp, 58° C.), Twist CTT TCC GCC CAC CCA CTT CCT CTT (SEQ ID NO:73) and GTC CAC GGG CCT GTC TCG CTT TCT (SEQ ID NO:74) (334 bp, 57° C.), and WntI TGA CAA CAT CGA TTT TGG TCG (SEQ ID NO:75) and GGC GAT TTC TCG AAG TAG ACG (SEQ ID NO:76) (375 bp, 58° C.). Positive control tissues included 12.5 dpc neural tube (Mash-1, Nestin, NgnI-3, Olig2, p75, Pax3, Pax4, Slug, Snail, SoxI-3, SoxI0, Twist, Wnt-I), undifferentiated ES cells or ES sphere colonies (E-cadherin, Nanog, Oct4), primary pancreas tissue (Beta2/NeuroD, GCK, Glut2, Hlxb9, Ins2, Isl-I, Nkx2.2, Nkx6.1, Pax6, PDX-I), embryonic mesoderm (Brachyury, GATA-I), embryonic endoderm (GATA-4, Hnf3β) and differentiated adult neurospheres (β₃-tubulin, MAP2, GFAP).

All publications, patents and patent applications are herein incorporated by reference in their entirety to the same extent as if each individual publication, patent or patent application was specifically and individually indicated to be incorporated by reference in its entirety.

The present invention has been described in terms of particular embodiments found or proposed by the present inventors to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### REFERENCES

1. Lechner, A. & Habener, J. F. Stem/progenitor cells from adult tissues: potential for the treatment of diabetes mellitus. Am. J. Physiol. Endocrinol. Metab. 284, E259-E266 (2003).
2. Ramiya, V. K. et al. Reversal of insulin-dependent diabetes using islets generated in vitro from pancreatic stem cells. Nat. Med. 6, 278-282 (2000).
3. Hunziker, E. & Stein, M. Nestin-expressing cells in the pancreatic islets of Langerhans. Biochem. Biophys. Res. Commun. 271, 116-119 (2000).
4. Zulewski, H. et al. Multipotential nestin-positive stem cells isolated from adult pancreatic islets differentiate ex vivo into pancreatic endocrine, exocrine, and hepatic phenotypes. Diabetes 50, 521-533 (2001).
5. Lendahl, U., Zimmerman, L. B. & McKay, R. D. CNS stem cells express a new class of intermediate filament protein. Cell 60, 585-595 (1990).
6. Weiss, S. et al. Is there a neural stem cell in the mammalian forebrain? Trends Neurosci. 19, 387-393 (1996).
7. Seaberg, R. M. & van der Kooy, D. Adult rodent neurogenic regions: the ventricular subependyma contains neural stem cells, but the dentate gyrus contains restricted progenitors. J. Neurosci. 22, 1784-1793 (2002).
8. Hadjantonakis, A.-K., Gertsenstein, M., Ikawa, M. Okabe, M. & Nagy, A. Generating green fluorescent mice by germline transmission of green fluorescent ES cells. Mech. Dev. 76, 79-90 (1998).
9. Morshead, C. M., Garcia, A. D., Sofroniew, M. V. & van der Kooy, D. The ablation of glial fibrillary acidic protein-positive cells from the adult central nervous system results in the loss of forebrain neural stem cells but not retinal stem cells. Eur. J. Neurosci. 18, 76-84 (2003).
10. Tropepe, V. et al. Retinal stem cells in the adult mammalian eye. Science 287, 2032-2036 (2000).
11. Li, H., Liu, H. & Heller, S. Pluripotent stem cells from the adult mouse inner ear. Nat. Med. 9, 1293-1299 (2003).
12. Jonsson, J., Carlsson, L. Edlund, T. & Edlund, H. Insulin-promoter-factor 1 is required for pancreas development in mice. Nature 371, 606-609 (1994).
13. Guz, Y. et al. Expression of murine STF-1, a putative insulin gene transcription factor, in beta cells of pancreas, duodenal epithelium and pancreatic exocrine and endocrine progenitors during ontogeny. Development 121, 11-8 (1995).
14. Kritzik, M. R. et al. PDX-1 and Msx-2 expression in the regenerating and developing pancreas. J. Endocrinol. 163, 523-530 (1999).
15. Zhang, Y.-Q. & Sarvetnick, N. Development of cell markers for the identification and expansion of islet progenitor cells. Diabetes Metab. Res. Rev. 19, 363-374 (2003).
16. Sommer, L., Ma, Q. & Anderson, D. J. Neurogenins, a novel family of atonal-related bHLH transcription factors, are putative mammalian neuronal determination genes that reveal progenitor cell heterogeneity in the developing CNS and PNS. Mol. Cell. Neurosci. 8, 221-241 (1996).
17. Bylund, M., Andersson, E., Novitch, B. G. & Muhr, J. Vertebrate neurogenesis is counteracted by Sox1-3 activity. Nat. Neurosci. 6, 1162-1168 (2003).
18. Guillemot, F. & Joyner, A. L. Dynamic expression of the murine achaete-scute homologue Mash-1 in the developing nervous system. Mech. Dev. 42,171-185 (1993).
19. Liu, Y. & Rao, M. Oligodendrocytes, GRPs and MNOPs. Trends Neurosci. 8, 410-412 (2003).
20. Rajagopal, J., Anderson, W. J., Kume, S., Martinez, O. I. & Melton, D. A. Insulin staining of ES cell progeny from insulin uptake. Science 299, 363 (2003).
21. MacDonald, P. E., El-Kholy, W., Riedel, M. J., Salapatek, A. M., Light, P. E. & Wheeler, M. B. The multiple actions of GLP-1 on the process of glucose-stimulated insulin secretion. Diabetes 51, S434-S442 (2002).
22. Roe, M. W. et al. Expression and function of pancreatic beta-cell delayed rectifier K⁺ channels. Role in stimulus-secretion coupling. J. Biol. Chem. 271, 32241-32246 (1996).
23. Ahren, B., Karlsson, S. & Lindskog, S. Cholinergic regulation of the endocrine pancreas. Prog. Brain Res. 84, 209-218 (1990).
24. Lardon, J., Rooman, I. & Bouwens, L. Nestin expression in pancreatic stellate cells and angiogenic endothelial cells. Histochem. Cell Biol. 117, 535-540 (2002).
25. Seaberg, R. M. & van der Kooy, D. Stem and progenitor cells: the premature desertion of rigorous definitions. Trends Neurosci. 26,125-131 (2003).
26. Mignone, J., Kukekov, V., Chiang, A. S., Steindler, D. & Enikolopov, G. Neural stem and progenitor cells in nestin-GFP transgenic mice. J. Comp. Neurol. 469, 311-324 (2004).
27. Sawamoto, K. et al. Generation of dopaminergic neurons in the adult brain from mesencephalic precursor cells labeled with a nestin-GFP transgene. J. Neurosci. 21, 3895-3903 (2001).
28. Weissman, I. L. Stem cells: Units of development, units of regeneration, and units in evolution. Cell 100,157-168 (2000).
29. Nichols, J. et al. Formation of pluripotent stem cells in the mammalian embryo depends on the POU transcription factor Oct4. Cell 95, 379-391 (1998).
30. Scholer, H. R., Dressier, G. R., Balling, R., Rohdewohid, H. & Gruss, P. Oct-4: a germline-specific transcription factor mapping to the mouse t-complex. EMBO J. 9, 2185-2195 (1990).
31. Chambers, I. et al. Functional expression cloning of Nanog, a pluripotency sustaining factor in embryonic stem cells. Cell 113, 642-655 (2003).
32. Gussoni, E. et al. Dystrophin expression in the mdx mouse restored by stem cell transplantation. Nature 401, 390-394 (1999).
33. Spangrude, G. J., Heimfeld, S. & Weissman, I. L. Purification and characterization of mouse hematopoietic stem cells. Science 241, 58-62 (1988).
34. Eftimie, R., Brenner, H., & Buonanno, A. (1991). Myogenin and MyoD join a family of skeletal muscle genes regulated by electrical activity. Proc. Natl. Acad. Sci. USA 88, 1349-1353 (1991).
35. Toma, J. G. et al: Isolation of multipotent adult stem cells from the dermis of mammalian skin. Nat. Cell. Biol. 3, 778-784 (2001).
36. McKenzie, I. et al. Evidence that skin-derived precursor cells (skps) are an endogenous neural crest-derived precursor. Soc. Neurosci. Abst. 28.9 (2003).
37. Goulding, M. D., Chalepakis, G., Deutsch, U., Erselius, J. R. & Gruss, P. Pax-3, a novel murine DNA binding protein expressed during early neurogenesis. EMBO J. 10, 1135-1147 (1991).
38. Stoetzel, C., Weber, B., Bourgeois, P., Bolcato-Bellemin, A. L. & Perrin-Schmitt, F. Dorso-ventral and rostro-caudal sequential expression of M-twist in the postimplantation murine embryo. Mech. Dev. 51, 251-263 (1995).
39. Honore, S. M., Aybar, M. J. & Mayor, R. Sox10 is required for the early development of the prospective neural crest in Xenopus embryos. Dev. Biol. 260, 79-96 (2003).
40. Lee, H. Y. et al. Instructive role of Wnt/beta-catenin in sensory fate specification in neural crest stem cells. Science 303, 1020-1023 (2004).
41. Aybar, M. J., Nieto, M. A. & Mayor, R. Snail precedes slug in the genetic cascade required for the specification and migration of the Xenopus neural crest.
   Development 130, 483-494 (2003).
42. Morrison, S. J., White, P. M., Zock, C. & Anderson, D. J. Prospective identification, isolation by flow cytometry, and in vivo self-renewal of multipotent mammalian neural crest stem cells. Cell 96, 737-749 (1999).
43. Peterson, D. A., Leppert, J. T., Lee, K.-F. & Gage, F. H. Basal forebrain neuronal loss in mice lacking neurotrophin receptor p75. Science 277, 837-838 (1997).
44. Scharfmann, R. Neurotrophin and neurotrophin receptors in islet cells. Horm. Metab. Res. 29, 294-296.
45. Lumelsky, N. et al. Differentiation of embryonic stem cells to insulin-secreting structures similar to pancreatic islets. Science 292, 1389-1394 (2001).
46. Persson-Sjogren, S., Zashihin, A. & Forsgren, S. Nerve cells associated with the endocrine pancreas in young mice: An ultrastructural analysis of the neuroinsular complex type I. Histochem. J. 33, 373-378 (2001).
47. Benveniste, P., Cantin, C., Hyam, D. & Iscove, N. N. Hematopoietic stem cells engraft in mice with absolute efficiency. Nat. Immunol. 4, 708-713 (2003).
48. Antonchuk, J., Sauvageau, G. & Humphries, R. K. HOXB4-induced expansion of adult hematopoietic stem cells ex vivo. Cell 109, 3945 (2002).
49. Hitoshi, S. et al. Notch pathway molecules are essential for the maintenance, but not the generation, of mammalian neural stem cells. Genes Dev. 16, 846-868 (2002).
50. Murtaugh, L. C., Stanger, B. Z., Kwan, K. M. & Melton, D. A. Notch signaling controls multiple steps of pancreatic differentiation. Proc. Natl. Acad. Sci. USA 100, 14920-14925.
51. Jiang, Y. et al. Pluripotency of mesenchymal stem cells derived from adult marrow. Nature 418, 41-49 (2002).
52. Clarke, D. L. et al. Generalized potential of adult neural stem cells. Science 288, 1660-1663 (2000).
53. Morshead, C. M., Benveniste, P., Iscove, N. N. & van der Kooy, D. Hematopoietic competence is a rare property of neural stem cells that may depend on genetic and epigenetic alterations. Nat. Med. 8, 268-273 (2002).
54. Piper, K., Ball, S. G., Turnpenny, L. W., Brickwood, S., Wilson, D. I. & Hanley, N. A. Beta-cell differentiation during human development does not rely on nestin-positive precursors: implications for stem cell-derived replacement therapy. Diabetologia 45, 1045-1047 (2002).
55. Humphrey, R. K. et al. Characterization and isolation of promoter-defined nestin-positive cells from the human fetal pancreas. Diabetes 52, 2519-2525 (2003).
56. Treutelaar, M. K. et al. Nestin-lineage cells contribute to the microvasculature but not endocrine cells of the islet. Diabetes 52, 2503-2512 (2003).
57. Delacour, A., Nepote, V., Trumpp, A. & Herreta, P. L. Nestin expression in pancreatic exocrine cell lineages. Mech. Dev. 121, 3-14 (2004).
58. Esni, F., Stoffers, D. A., Takeuchi, T. & Leach, S. D. Origin of exocrine pancreatic cells from nestin-positive precursors in the developing mouse pancreas. Mech. Dev. 121, 15-25 (2004).
59. Klein, T., Ling, Z., Heimberg, H., Madsen, 0. D., Heller, R. S., Serup, P. Nestin is expressed in vascular endothelial cells in the adult human pancreas. J. Histochem. Cytochem. 51, 697-706 (2003).
60. Selander, L. & Edlund, H. Nestin is expressed in mesenchymal and not epithelial cells of the developing mouse pancreas. Mech. Dev. 113, 189-192 (2002).
61. Polesskaya, A., Seale, P. & Rudnicki, M. A. Wnt signaling induces the myogenic specification of resident CD45+ adult stem cells during muscle regeneration. Cell 113, 841-852 (2003).
62. Gotoh, M., Maki, J., Kiyoizumi, T., Satomi, S. & Monaco, A. P. An improved method for isolation of mouse pancreatic islets. Transplantation 40, 437-438 (1985).
63. Tropepe, V. et al. Distinct neural stem cells proliferate in response to EGF and FGF2 in the developing mouse telencephalon. Dev. Biol. 208, 166-188 (1999).
64. Kang, G. et al. Epac-selective camp analog 8-pCPT-2′-O-Me-cAMP as a stimulus for Ca²⁺-induced Ca²⁺ release and exoctyosis in pancreatic β-cells. J. Biol. Chem. 278, 8279-8285 (2003).

65. Smukler, S. R., Tang, L., Wheeler, M. B. & Salapatek, A. M. F. Exogenous nitric oxide and endogenous glucose-stimulated β-cell nitric oxide augment insulin release. Diabetes 51, 3450-3460.

TABLE 1
Comparison of the mean percentages of neural and pancreatic cell progeny generated from murine
adult pancreas (PSC) colonies and adult forebrain-derived neurospheres (mean % ± SEM).

| | Neural Cell Types | | | | | | | | |
|---|---|---|---|---|---|---|---|---|---|
| | | | Oligo | Pancreatic Cell Types | | | | | Stellate/Neural |
| | Neurons | Astrocytes | dendrocytes | β-Cells | α-cells | δ-cells | Acinar Cells | Stellate Cells | Cells** |
| | (β₃-tubulin⁺) | (GFAP⁺) | (O4⁺) | (insulin⁺) | (glucagon⁺) | (somatostatin⁺) | (amylase⁺) | (SMA⁺) | (nestin⁺) |
| PSC | 26.4 ± 3.8* | 7.4 ± 1.3* | 2.4 ± 0.7 | 4.7 ± 1.0* | 6.3 ± 2.0* | 4.5 ± 0.6* | 6.2 ± 1.2* | 57.4 ± 7.0* | 49.6 ± 2.9* |
| Colony | | | | | | | | | |
| Forebrain | 3.7 ± 0.6 | 84.2 ± 1.4 | 4.3 ± 1.7 | 0.0 ± 0.0 | 0.0 ± 0.0 | 0.0 ± 0.0 | 0.0 ± 0.0 | 0.0 ± 0.0 | 9.5 ± 1.8** |
| Neuro- | | | | | | | | | |
| sphere | | | | | | | | | |

Summary table illustrating the fundamental differences in cell lineage potential of clonal colonies generated from the adult forebrain or adult pancreas. The numbers of each cell type are expressed us a percentage of DAPI-positive nuclei.
*indicates a significant difference in the proportion of the indicated cell type generated by pancreas colonies compared to neurospheres (p < 0.05). Interestingly, PSC colonies generate a significantly higher proportion of neurons than adult brain-derived neurospheres, but significantly fewer astrocytes. Pancreatic cell types were detected only in PSC colonies and were not detected in differentiated brain-derived neurospheres.
**Pancreatic stellate cells are known to express both SMA and nestin, and both of these markers were expressed in PSC progeny with characteristic large, flat morphology. However, because there was no SMA expression in neurosphere cells, the nestin immunoreactivity in these cultures likely represents a subpopulation of undifferentiated neural precursors. Further, nestin and β₃-tubulin are sometimes co-expressed as newly-generated neurons differentiate.

TABLE 2
Comparison of the gene expression profile of PSC colonies
and brain-derived neurospheres by RT-PCR analysis, for
both undifferentiated and differentiated conditions.

| | PSC Colony | | Brain-derived Neurosphere | |
|---|---|---|---|---|
| | Undiffer- | Differ- | Undiffer- | Differ- |
| | entiated | entiated | entiated | entiated |
| β₃-tubulin | nd | + | + | + |
| Beta2/NeuroD | nd | + | + | − |
| Brachyury | − | nd | − | − |
| GATA-1 | − | nd | − | − |
| GATA-4 | − | nd | − | − |
| GCK | nd | + | − | nd |
| GFAP | − | + | − | + |
| Glut2 | − | + | − | nd |
| Hlxb9 | nd | + | − | − |
| HNF3β | − | nd | − | − |
| Ins2 | − | + | − | nd |
| Isl-1 | nd | + | − | − |
| Mash-1 | + | nd | + | + |
| Nanog | − | − | − | − |
| Nestin | + | nd | + | nd |
| Ngn1 | − | nd | + | − |
| Ngn2 | − | − | − | − |
| Ngn3 | + | nd | + | − |
| Nkx2.2 | nd | + | + | + |
| Nkx6.1 | nd | + | − | − |
| Oct4 | − | nd | − | nd |
| Olig2 | − | nd | + | nd |
| Pax3 | − | nd | + | − |
| Pax4 | − | nd | + | − |
| Pax6 | nd | + | + | + |
| p48/Ptf1 | − | nd | − | − |
| p75 | +/− | nd | + | + |
| Slug | + | nd | − | nd |
| Snail | + | nd | − | nd |
| Sox1 | − | nd | + | nd |
| Sox2 | + | nd | + | + |
| Sox3 | + | nd | + | + |
| Sox10 | − | nd | − | nd |
| Twist | − | nd | +/− | +/− |
| Wnt1 | − | nd | − | − |

Summary table illustrating the differences in gene expression of both differentiated and undifferentiated clonal colonies generated from the adult forebrain or adult pancreas.
+, the mRNA was reliably detected;
+/−, the mRNA was detected in some but not all samples,
−, the mRNA was reliably not detected;
nd, not determined.

1. Clonal pancreatic stem cells isolated from the pancreas of a mammal.

2. Clonal pancreatic stem cells, pancreatic cells and/or neural cells produced from the cells of claim 1.

3. The cells of claim 2, wherein the pancreatic cells comprise at least one of alpha cells, beta cells, delta cells, acinar cells and stellate cells.

4. The cells of claim 2, wherein the neural cells comprise at least one of astrocytes, oligodendrocytes, and neurons.

5. The cells of claim 1, which proliferate in the presence of growth factors, Wnt signaling activators or Notch signaling activators.

6. The cells of claim 5, wherein the growth factors comprise EGF and FGF2.

7. The cells of claim 5, wherein the Wnt signaling activator comprises BIO.

8. A method for producing clonal stem cell populations from a pancreatic tissue of a mammal, comprising:
a) dissociating all or part of the tissue into single cells,
b) culturing the cells for a time period sufficient that each proliferative pancreatic stem cell has repeatedly divided to produce a corresponding clonal cell population.

9. The method of claim 8, further comprising differentiating the cells of one of the clonal stem cell populations into pancreatic cells and/or neural cells.

10. The method of claim 9, comprising, prior to the differentiating step, culturing the clonal stem cell population in the presence of growth factors, Wnt signaling activators and/or Notch signaling activators.

11. The method of claim 8, wherein the pancreatic cells comprise at least one of alpha cells, beta cells, delta cells, acinar cells and stellate cells

12. The method of claim 8, wherein the neural cells comprise at least one of astroyctes, oligodendrocytes, and neurons.

13. A method of prophylaxis or treatment of a disease, disorder or abnormal state of the pancreas in a subject, comprising administering to the subject the pancreatic stem cells or pancreatic cells of claim 2.

14. The method of claim 13, wherein the disease, disorder or abnormal physical state is selected from the group consisting of type I or type II diabetes, pancreatitis, pancreatic degeneration and cancers of the pancreas.

15. The method of claim 14, further comprising implanting the cells into the pancreas, liver or kidney of the subject.

16. A method of treating diabetes or other disease, disorder or abnormal physical state of the pancreas comprising differentiating pancreatic stem cells into beta cells and administering the beta cells to a subject, wherein the beta cells secrete insulin in response to glucose.

17. A method of prophylaxis or treatment of a disease, disorder or abnormal physical state of the nervous system in a subject, comprising administering to the subject the pancreatic stem cells or neural cells of claim 2.

18. A pharmaceutical composition comprising the cells of claim 2 and a pharmaceutically acceptable carrier, diluent or excipient.

19. A method for identifying a compound for modulating a beta cell to increase or reduce insulin secretion by the beta cell, comprising
introducing the compound to a cell culture comprising beta cells differentiated from the cells of claim 1,
contacting the beta cells with glucose, and
determining whether the beta cells secrete increased or reduced insulin in response to the glucose, wherein increased insulin secretion indicates that the compound increases insulin secretion by the beta cell and reduced insulin secretion indicates that the compound reduces insulin secretion by the beta cell.
